(19) United States
(12) Patent Application Publication
(10) Pub. No.: US 2020/0166524 A1
(43) Pub. Date: May 28, 2020

(54) EXTRACELLULAR VESICLE MARKERS FOR STABLE ANGINA AND UNSTABLE ANGINA

(71) Applicant: UMC UTRECHT HOLDING B.V., Utrecht (NL)

(72) Inventors: Dominicus Paschalis Victor DE KLEIJN, Wijk bij Duurstede (NL); Leonardus TIMMERS, Utrecht (NL)

(21) Appl. No.: 16/060,155

(22) PCT Filed: Dec. 7, 2016
(86) PCT No.: PCT/EP2016/080131

§ 371 Date: Jun. 7, 2018

(30) Foreign Application Priority Data
Dec. 8, 2015 (NL) 2015924

(51) Int. Cl. G01N 33/68

(52) U.S. Cl. CPC G01N 33/6893; G01N 2800/324; G01N 2333/8139; G01N 2333/70596; G01N 2333/8121

(57) ABSTRACT

The present invention relates to the determination of protein markers associated with extracellular vesicles present in sub-fractions of plasma samples taken from people that experience chest pain and are suspected to experience ischemic heart disease. The invention relates to the use of the markers in the identification of subjects suffering from, or at risk of suffering from, an ischemic heart disease, in particular stable angina and unstable angina. Especially preferred markers are SerpinC1, SerpinG1, CD14, Cystatin C and SerpinF2.

### FIELD OF THE INVENTION

The invention relates to the field of medicine. More in particular it relates to a method for identifying a subject suffering from, or being at risk of suffering from, an ischemic heart disease by measuring the concentration or a value related thereto of one or more protein markers in one or more blood plasma fractions. The protein markers are especially suitable for identifying a subject suffering from, or being at risk of suffering from unstable and stable angina, and equivalents thereof.

### BACKGROUND ART

Ischemic heart disease is a major cause of morbidity and mortality worldwide. Although therapy for ischemic heart disease has greatly improved and mortality has gradually declined in industrialized countries during the last decades, mortality from ischemic heart disease is still rising in other parts of the world such as Africa and parts of Asia (Mathers and Loncar. Projections of global mortality and burden of disease from 2002 to 2030. PLoS Med. 2006; 3:e442; WHO. World Health Statistics 2013. Geneva, Switzerland; Alwan A. WHO. Global status report on noncommunicable diseases 2010. Geneva, Switzerland). Clinical syndromes associated with ischemic heart disease are stable angina, unstable angina and myocardial infarction.

### Stable Angina

Stable coronary artery disease is the underlying cause of the clinical syndrome referred to as ‘stable angina’: episodes of stress-, exercise- or emotion-induced chest pain. Stable coronary artery disease can also cause atypical symptoms such as shortness of breath or reduced exercise tolerance. These symptoms are not generally referred to as stable angina, but can be referred to as ‘stable angina equivalents’ as they are caused by the same underlying disorder. Stable coronary artery disease is generally characterized by episodes of reversible myocardial demand/supply mismatch due to significant stenosis in the coronary arteries. Because the appearance of chest pain or equivalent symptoms are in a sense predictable, the disease is referred to as stable angina. The terms ‘stable angina’ and ‘stable coronary artery disease’ are often mixed and often used in the art to mean the same thing. For clarity reasons, ‘stable angina’ as used herein refers to all symptoms (predictable episodes of stress-, exercise- or emotion-induced chest pain, and atypical symptoms such as shortness of breath or reduced exercise tolerance) that are caused by stable coronary artery disease. The prevalence of stable angina varies from 5-14% depending on gender and age. Annual incidence of death is 1.2-2.4% (vs 0.6% in subjects without obstructive coronary artery disease), and the annual incidence of myocardial infarction is 2.7%. Because of the variety in clinical presentation and a broad differential diagnosis (such as musculoskeletal or psychological problems, pulmonary embolism, pneumonia, pneumothorax, pericarditis), the diagnosis of stable angina is notoriously challenging. Patients can be referred for exercise ECG or non-invasive imaging such as cardiac CT, MRI or nuclear scan. Such imaging tests have a sensitivity and specificity of around 85%, and are time consuming and expensive. In many cases, invasive coronary angiogram is needed to confirm the diagnosis and to determine the options for revascularisation by percutaneous coronary intervention or coronary artery bypass grafting. The diagnostic work-up is inefficient and expensive. A rapid straightforward test for diagnosing (or ruling out) stable angina is currently non-existent.

### Unstable Angina

Annually, millions of patients enter the emergency rooms with chest pain or other symptoms that are suggestive of Acute Coronary Syndrome (ACS). ACS is a life threatening condition mostly caused by intracoronary thrombus formation leading to acute luminal narrowing or even occlusion. There are two types of ACS: unstable angina and myocardial infarction. Myocardial infarction can be quickly diagnosed on ECG and/or elevation of cardiac troponin in the blood. Unstable angina is an unstable coronary syndrome, without signs of myocardial injury (such as an elevated level of cardiac troponin). The term unstable is used because—in contrast to stable angina—the chest pain is not so much predictable and can also occur in rest and generally increases in severity over time. 5% of all people entering the emergency rooms with chest pain or other symptoms that suggest ACS, have unstable angina. Rapid diagnosis of unstable angina is essential, since it is associated with a high risk of adverse cardiac events; more than in the case of stable angina. About 35% of unstable angina patients undergo revascularisation during the index visit. Importantly, missed unstable angina patients that are sent home, often return for revascularisation within one year (˜50%), or suffer myocardial infarction (8% in 30 days; MINERVA database Meander MC Amersfoort). As for stable angina, the diagnosis for unstable angina is similarly challenging. Troponin is negative per definition and the ECG is diagnostic in only 10-15% of cases. Hospitalisation is often required for determining serial cardiac troponin levels, non-invasive testing such as exercise ECG or imaging, or for an invasive coronary angiogram. No rapidly determinable and reliable diagnostic markers are available that positively identify the unstable angina patient, or that will on the other hand, exclude unstable angina.

### Blood Biomarkers

Clearly, as outlined above, the establishment of a timely identification of a subject suffering from, or being at risk of suffering from, stable angina and unstable angina is important to allow adequate treatment. An accurate early diagnosis may take away the symptoms and improve the prognosis of the patients. In addition, ruling out both diseases prevents unnecessary referrals and hospital admissions for time consuming non-invasive and invasive diagnostic testing, thereby significantly decreasing the health care burden. A simple and rapid test for all patients presenting with symptoms suggestive of stable and unstable angina is highly desired. It has been recognized in the art that a blood test is the most convenient, since it is a low risk diagnostic tool and easily accessible in both primary and secondary care.

Many proteins have been investigated in relation to stable and unstable angina. The inflammatory concept of atherosclerosis led many investigators to concentrate on inflammatory mediators such as hsCRP, GDF-15, neopterin, IL-6, IL-10, IL-17, MPO, procalcitonin, Fetuin A, Lp-PLA2, and MMPs/TIMPs. Although these markers have some prognostic value, none of them appear to have sufficient diagnostic power to discriminate patients with stable and unstable angina (Tsaknis et al. Clinical usefulness of novel serum and imaging biomarkers in risk stratification of patients with stable angina. Dis Markers 2014:831364).

Also non-protein markers have been investigated. In a small pilot study involving 53 patients, three micro RNAs (miRNAs) appeared to have the potential to discriminate patients with stable angina from controls. MiRNAs are small non-coding RNAs that regulate complex biological processes. The miRNAs that were found were miR-1, miR-126, miR-483-5p, having an Area Under the Curve (AUC) of 0.91, 0.92 and 0.85 respectively (D'Alessandra et al. Diagnostic potential of plasmatic MicroRNA signatures in stable and unstable angina. PLoS One. 2013 Nov. 15: 8). However, these miRNA have thus far not been validated. In the same pilot study, three potential biomarkers for unstable angina were also found: miR-1, miR-126 and miR-133a, with AUC's of 0.92, 0.87 0.91 respectively. In contrast however, these miRNAs were not identified as potential biomarkers for unstable angina in other studies. In yet another investigation, a miRNA panel (consisting of miR-132, miR-150 and miR-186) showed the highest discriminating power with an AUC of 0.91 (Zeller et al. Assessment of microRNAs in patients with unstable angina pectoris. Eur Heart J. 2014 Aug. 14; 35(31):2106-14). It is evident that miRNAs could potentially be used as biomarkers for stable and unstable angina, but additional studies are required to validate their potential and to address inconstancies between the different studies. Next to this, detection of miRNA is technically challenging requiring cDNA synthesis and qPCR which limits their application in an acute setting (such as with unstable angina) or in GP settings.

It is concluded that there remains an urgent need for alternative circulating biomarkers to identify subjects suffering from, or being at risk of suffering from, an ischemic heart disease and to discriminate between life-threatening events such as stable and unstable angina of patients with chest pain or equivalent symptoms on the one hand, and milder events in people that also experience similar symptoms but do not suffer from such an ischemic heart disease, on the other hand.

### SUMMARY OF THE INVENTION

The present invention relates to a method for identifying a subject suffering from, or being at risk of suffering from, an ischemic heart disease, said method comprising the steps of:
a) obtaining one or more of the plasma fractions selected from the group consisting of the Low-Density Lipoprotein (LDL) fraction, the High-Density Lipoprotein (HDL) fraction, the remaining (REX) fraction and the total (TEX) fraction from a plasma sample of said subject;
b) determining one or more values, wherein each of the one or more values:
   is derived from a concentration of a protein marker in one of said plasma fractions, wherein the protein marker is selected from the group consisting of SerpinC1, Cystatin C, CD14, SerpinF2 and SerpinG1, and/or
   is a ratio of two values derived from the concentrations of a single protein marker in two different ones of said plasma fractions, wherein the protein marker is selected from the group consisting of SerpinC1, Cystatin C, CD14, SerpinF2 and SerpinG1,
c) performing a comparison of the one or more values as determined in step b) with one or more corresponding reference values, which has been derived in the same way from the concentration of the same one or more protein marker in corresponding plasma fractions as determined in a group of reference subjects not suffering from ischemic heart disease, wherein a statistically significant difference between the one or more values determined in step b) and the one or more corresponding reference values is indicative of the subject suffering, or being at risk of suffering from, an ischemic heart disease.

By this method it is for the first time possible to identify subjects suffering from an ischemic heart disease, such as unstable angina or stable angina, from a blood sample with a high level of statistical significance.

In a preferred embodiment, said ischemic heart disease is unstable angina and at least one of the one or more values in step b) is selected from the group consisting of values derived from the concentration of:
SerpinC1 in the HDL plasma fraction,
CD14 in the HDL plasma fraction,
CD14 in the REX plasma fraction,
CD14 in the TEX plasma fraction, and
SerpinF2 in the TEX plasma fraction,
   and/or wherein at least one of the one or more values in step b) is a ratio of two values derived from concentrations of a single protein marker in two different ones of said plasma fractions, selected from the group consisting of:
the concentration of SerpinC1 in the LDL plasma fraction over the concentration in the HDL plasma fraction,
the concentration of SerpinC1 in the HDL plasma fraction over the concentration in the REX plasma fraction,
the concentration of SerpinC1 in the HDL plasma fraction over the concentration in the TEX plasma fraction,
the concentration of CD14 in the LDL plasma fraction over the concentration in the HDL plasma fraction,
the concentration of CD14 in the LDL plasma fraction over the concentration in the TEX plasma fraction,
the concentration of CD14 in the HDL plasma fraction over the concentration in the REX plasma fraction,
the concentration of CD14 in the HDL plasma fraction over the concentration in the TEX plasma fraction,
the concentration of Cystatin C in the LDL plasma fraction over the concentration in the HDL plasma fraction,
the concentration of Cystatin C in the HDL plasma fraction over the concentration in the REX plasma fraction,
the concentration of Cystatin C in the HDL plasma fraction over the concentration in the TEX plasma fraction, and
the concentration of SerpinF2 in the REX plasma fraction over the concentration in the TEX plasma fraction.

This embodiment of the invention provides for the first time a method to identify subjects suffering from, or being at risk of suffering from, unstable angina from a blood sample with a high level of statistical significance.

In another preferred embodiment, said ischemic heart disease is stable angina, and at least one of the one or more values in step b) is selected from the group consisting of values derived from the concentration of:
SerpinC1 in the HDL plasma fraction,
SerpinC1 in the TEX plasma fraction, and
CD14 in the HDL plasma fraction,
and/or wherein at least one of the one or more values is a ratio of two values derived from concentrations of a single protein marker in two different ones of said plasma fractions, selected from the group consisting of:
   the concentration of SerpinC1 in the LDL plasma fraction over the concentration in the HDL plasma fraction,
   the concentration of SerpinC1 in the HDL plasma fraction over the concentration in the REX plasma fraction,
   the concentration of SerpinC1 in the HDL plasma fraction over the concentration in the TEX plasma fraction,
   the concentration of SerpinC1 in the REX plasma fraction over the concentration in the TEX plasma fraction,
   the concentration of CD14 in the LDL plasma fraction over the concentration in the HDL plasma fraction,
   the concentration of CD14 in the HDL plasma fraction over the concentration in the REX plasma fraction,
   the concentration of CD14 in the HDL plasma fraction over the concentration in the TEX plasma fraction,
   the concentration of Cystatin C in the LDL plasma fraction over the concentration in the HDL plasma fraction,
   the concentration of Cystatin C in the HDL plasma fraction over the concentration in the REX plasma fraction,
   the concentration of Cystatin C in the HDL plasma fraction over the concentration in the TEX plasma fraction,
   the concentration of SerpinF2 in the LDL plasma fraction over the concentration in the HDL plasma fraction,
   the concentration of SerpinF2 in the LDL plasma fraction over the concentration in the REX plasma fraction,
   the concentration of SerpinF2 in the HDL plasma fraction over the concentration in the TEX plasma fraction, and
   the concentration of SerpinG1 in the REX plasma fraction over the concentration in the TEX plasma fraction.
   This embodiment of the invention provides for the first time a method to identify subjects suffering from, or being at risk of suffering from, stable angina from a blood sample with a high level of statistical significance.
   In a further embodiment, the present invention relates to a kit comprising means for performing the method according to the invention.

### BRIEF DESCRIPTION OF THE DRAWINGS

These and other aspects of the invention are apparent from and will be elucidated with reference to the embodiments described hereinafter.

FIG. 1 is a table showing the baseline of 30 unstable angina cases and 30 matched controls.

FIG. 2 is a table showing the measurement of CD14, SerpinG1, SerpinF2, Cystatin C and SerpinC1 levels in each of the ten density gradient fractions per plasma sub-fraction.

FIG. 3 displays the Receiver Operating Characteristic (ROC) plot of the identification of unstable angina in an Emergency Department cohort of chest pain patients of 30 definitive unstable angina and 30 matched controls using the combination of markers consists of SerpinC1-HDL+CD14-TEX+SerpinC1-LDL.

FIG. 4 shows a ROC plot of the identification of stable angina (here referred to as stable coronary artery disease (SCAD)) in a cohort of suspected symptomatic coronary artery disease of 30 definitive cases with stable angina and 30 matched controls using the combination of markers consists of SerpinC1-HDL+SerpinG1-TEX+SerpinC1-REX.

FIG. 5 shows the highest scoring combinations of three marker/fraction and/or marker/ratio in the Myomarker cohort (two columns per page, each column showing the marker combination on the left and its AUC value on the right). Abbreviations: c1=SerpinC1, g1=SerpinG1, f2=SerpinF2, cc=Cystatin C, cd14=CD14, Id1=Low-Density lipoprotein, hd1=High-Density Lipoprotein, rex=remaining fraction, tex=total fraction. Whenever the abbreviation of two plasma fractions are mentioned in connection with a protein marker, it means that the ratio is taken between the concentration (or a value derived therefrom) in these two plasma fractions. By way of example, “c1ld1hdl” means the ratio of a value derived from the concentration of SerpinC1 in fraction LDL over HDL. In total the list contained 19,600 combinations, only the combinations with an AUC of 0.876 or higher are shown.

FIG. 6 shows the highest scoring combinations of three marker/fraction and/or marker ratio pairs in the Minerva cohort (two columns per page, each column showing the marker combination on the left and its AUC value on the right). Abbreviations are as in FIG. 5 and so is the nomenclature for when two plasma fractions are mentioned in connection with a protein marker. In total the list contained 19,600 combinations, only the combinations with an AUC of 0.9 or higher are shown. The best scoring combination of marker/fraction pairs (i.e. comprising no marker/ratio pairs) SerpinC1-HDL+CD14-TEX+SerpinC1-LDL (AUC 0.933) depicted in FIG. 3 is underlined.

### DETAILED DESCRIPTION OF EMBODIMENTS

The present invention relates to a method for identifying a subject suffering from, or being at risk of suffering from, an ischemic heart disease from a blood sample based on the concentration (or a value derived from the concentration) of one or more identified protein markers in one or more blood plasma fractions or based on the ratio of concentration (or values derived from the concentration) of a given protein marker in two different ones of said plasma fractions and comparing it to the corresponding concentration and/or ratio as determined in a group of reference subjects not suffering from ischemic heart disease, wherein a statistically significant difference between the concentration and/or ratio determined for the subject in question and the corresponding concentration and/or ratio of the reference group is indicative of the subject suffering, or being at risk of suffering, from an ischemic heart disease. In the context of the present invention, “concentration” of a given protein marker in a given plasma sub-fraction can also mean a “value derived from the concentration” of the protein marker in the plasma sub-fraction in question, wherein a value derived from the concentration is a value that directly correlates with the concentration of the protein marker. In one embodiment, the present invention relates to a method for identifying a subject suffering from, or being at risk of suffering from, an ischemic heart disease, such as unstable angina and stable angina, said method comprising the steps of:
a) obtaining one or more of the plasma fractions selected from the group consisting of the Low-Density Lipoprotein (LDL) fraction, the High-Density Lipoprotein (HDL) fraction, the remaining (REX) fraction and the total (TEX) fraction from a plasma sample of said subject;
b) determining one or more values, wherein each of the one or more values:
   is derived from a concentration of a protein marker in one of said plasma fractions, wherein the protein marker is selected from the group consisting of SerpinC1, Cystatin C, CD14, SerpinF2 and SerpinG1, and/or
   is a ratio of two values derived from the concentrations of a single protein marker in two different ones of said plasma fractions, wherein the protein marker is selected from the group consisting of SerpinC1, Cystatin C, CD14, SerpinF2 and SerpinG1,
c) performing a comparison of the one or more values as determined in step b) with one or more corresponding reference values, which has been derived in the same way from the concentration of the same one or more protein marker in corresponding plasma fractions as determined in a group of reference subjects not suffering from ischemic heart disease, wherein a statistically significant difference between the one or more values determined in step b) and the one or more corresponding reference values is indicative of the subject suffering, or being at risk of suffering, from an ischemic heart disease.

By this method it is for the first time possible to identify subjects suffering from an ischemic heart disease, such as stable or unstable angina, from a blood sample with a statistical relevance characterized by that the one or more values in step b) are selected such that the area under the curve (AUC) differs from the diagonal reference line of 0.5 with a p-value of 0.05 or less, as determined by Receiver Operating Characteristic (ROC) plot analysis of the combination of said one or more values based on a suitable group of definitive subjects and group of reference subjects. Preferably, the statistical relevance of the method according to the invention is characterized by that the one or more values in step b) are selected such that the area under the curve (AUC) is 0.8 or more, such as 0.85 or more, 0.9 or more or 0.95 or more and the p-value is 0.05 or less, as determined by Receiver Operating Characteristic (ROC) plot analysis of the combination of said one or more values based on a suitable group of definitive subjects and group of reference subjects. Even more preferably, the statistical relevance of the method according to the invention is characterized by resulting in a negative predictive value and/or a positive predictive value is 0.8 or more, such as 0.85 or more, 0.9 or more or 0.95 or more. In one embodiment, the protein marker is SerpinC1. The method according to the present invention is an in vitro method.

In the context of the present invention, the subject may be any mammal but is preferably a human subject and even more preferably a human patient, such as a human patient having chest pain. In the context of the present invention, the group of reference subjects is of the same origin as the subject itself, and accordingly, if the subject is a human, the group of reference subjects are also human. In one embodiment of the present invention, the group of reference subjects have the same clinical signs and symptoms as the subject itself, but are not suffering from ischemic heart disease.

The plasma fractions used in the present invention can be obtained as follows: Using dextrane sulphate and Mn, chylomicrons, Very Low-Density Lipoprotein (VLDL) and Low-Density Lipoprotein (LDL) are precipitated in the first fractionation step. Hereinafter, the plasma fraction obtained in this first fractionation step is called “LDL”. In the second step High-Density Lipoprotein (HDL) is precipitated. Sub-fractionation of the LDL and HDL fractions from plasma is known in the art. The remaining sub-fraction is referred to as (REX), is almost completely depleted of lipoprotein particles. The inventors of the present invention have investigated the protein content of plasma extracellular vesicles present in three different blood plasma sub-fractions: LDL, HDL and the remaining plasma subfraction REX. Besides that, the inventors have assessed the protein patterns associated with extracellular vesicles in unfractionated (total) plasma (referred to as TEX). The TEX fraction can be obtained by using Exoquick precipitation buffer sold by SBI or Xtractt buffer from Cavadis B.V. in accordance with manufacturers' instructions. In the context of the present invention, the term “plasma fraction” refers to the LDL, HDL, REX and TEX plasma fractions. The terms “plasma fraction” and “plasma sub-fraction” are used interchangeably herein.

Plasma extracellular vesicles are bilayer lipid membrane vesicles including exosomes, microvesicles and microparticles (Colombo et al. Biogenesis, secretion, and intercellular interactions of exosomes and other extracellular vesicles. Ann Rev Cell Dev Biol 2014:255-289). Exosomes are synthesized in the multivesicular endosome, while microvesicles are formed by the plasma membrane. Once secreted in the plasma these extracellular vesicles can no longer be distinguished from each other. This is why exosomes are often called microvesicles and microvesicles are often referred to as exosomes. For the sake of clarity, extracellular vesicles as used herein refer to all such extracellular bilayer lipid membrane vesicles present in the sub-fractions of the plasma, as outlined further below.

Extracellular vesicles play an important role in intercellular communication and contain or are associated with proteins, miRNAs and mRNA from the cell of origin, reflecting their physiological or pathological status. It is known that distinct bilayer membrane extracellular plasma vesicles co-fractionate with monolayer LDL. Other bilayer membrane extracellular vesicles (with a different content) co-fractionate with HDL (Zhang et al. Circulating TNFR1 exosome-like vesicles partition with the LDL fraction of human plasma. Biochem Biophys Res Comm 2008:579-584). This allows separation of distinct plasma extracellular vesicle sub-fractions via sequential LDL and HDL isolation. Through this, the inventors were able to identify subpopulations of extracellular vesicles, each with their own particular protein content and potentially (patho)-physiological pathways associated therewith. Plasma extracellular vesicles have been recognized in the art as having potential value in relation to cardiovascular disease (Wang et al. Plasma extracellular vesicle protein content for diagnosis and prognosis of global cardiovascular disease. Neth Heart J 2013:467-471).

In the context of the present invention, protein marker SerpinC1 is identified as UniProtKB—P01008 (ANT3_HUMAN), protein marker Cystatin C is identified as UniProtKB—P01034 (CYTC_HUMAN), protein marker CD14 is identified as UniProtKB—P08571 (CD14_HUMAN), protein marker SerpinF2 is identified as UniProtKB—P08697 (A2_AP_HUMAN) and protein marker SerpinG1 is identified as UniProtKB—P05155 (IC1_HUMAN), when the subject is a human, such as a human patient. The skilled person will understand, that if the subject is another mammal than human, the protein markers to be used in accordance with the invention, will be the corresponding proteins in the mammal in question.

In the context of the present invention, a value derived from the concentration of a given protein marker in a given plasma fraction is also called a value of a “marker/fraction pair”. For convenience, a marker/fraction pair is sometimes abbreviated herein by [name of the marker]-[name of the plasma fraction]. For example, the protein marker SerpinC1 determined in plasma fraction LDL interchangeably is also referred to by “SerpinC1 in LDL”, “SerpinC1-LDL” or “C1-LDL”. Likewise, a value which is a ratio of two values derived from the concentrations of a given protein marker in two different plasma fractions is called a value of a “marker/ratio pair”. For convenience, a marker/ratio pair is sometimes abbreviated herein by [name of the marker]-[name of plasma fraction 1]/[name of plasma fraction 2]. For example, the protein marker SerpinC1 determined in plasma fraction LDL and HDL and for which the ratio of the values derived from the concentrations in LDL over HDL is used, is also referred to by “ratio of SerpinC1 in LDL over HDL”, “SerpinC1-LDL/HDL” or “C1-LDL/HDL”. The value derived from the concentration of a given protein marker in a given plasma fraction can be determined in any way known to a person skilled in the art. In one embodiment of the present invention, the determination of the one or more values in step b) is performed by an immunoassay using antibodies specific to the one or more protein markers in question. The immunoassay can suitably be a beads-based immunoassay, wherein the beads are conjugated with the selected antibodies to synthesize the bead-capture antibody complex. The bead-capture antibody complex is then incubated with the samples and subsequently with biotinylated antibodies to detect the captured protein by reaction with streptavidin subsequent and quantification.

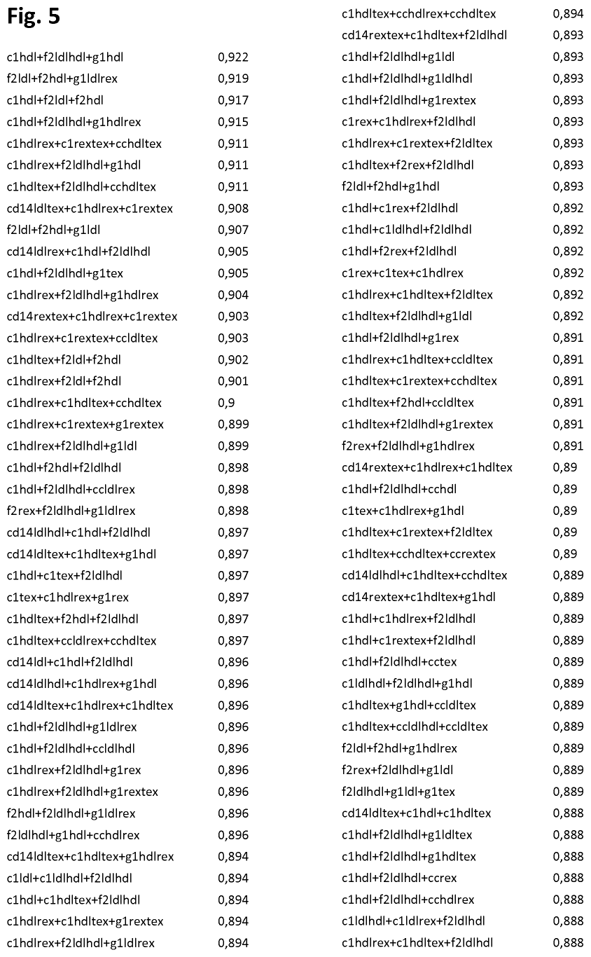

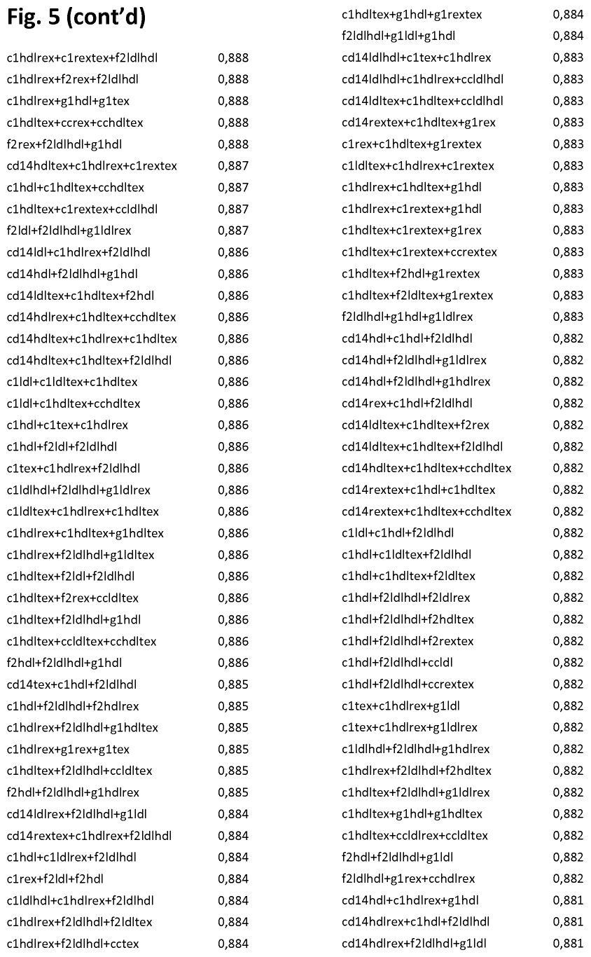

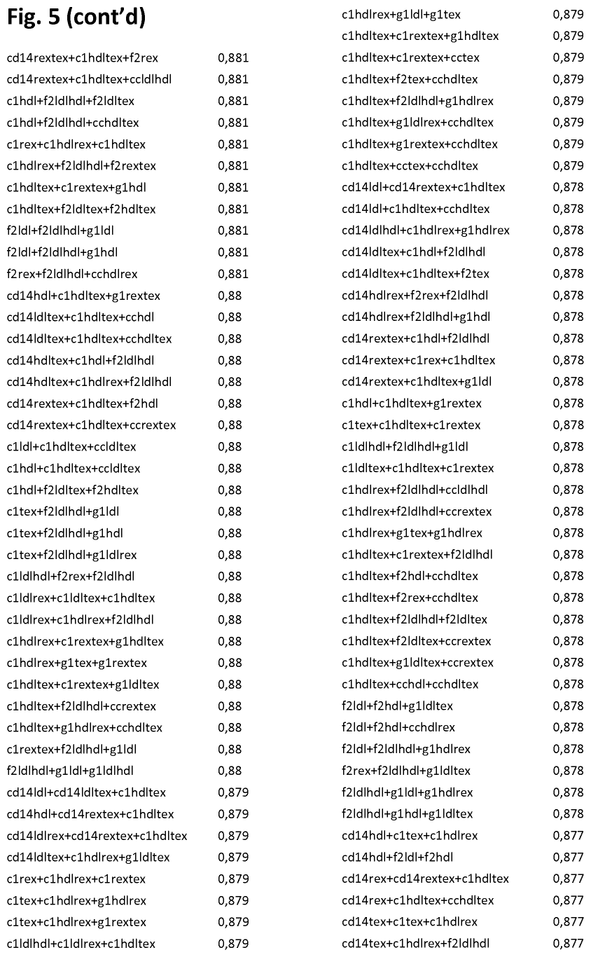

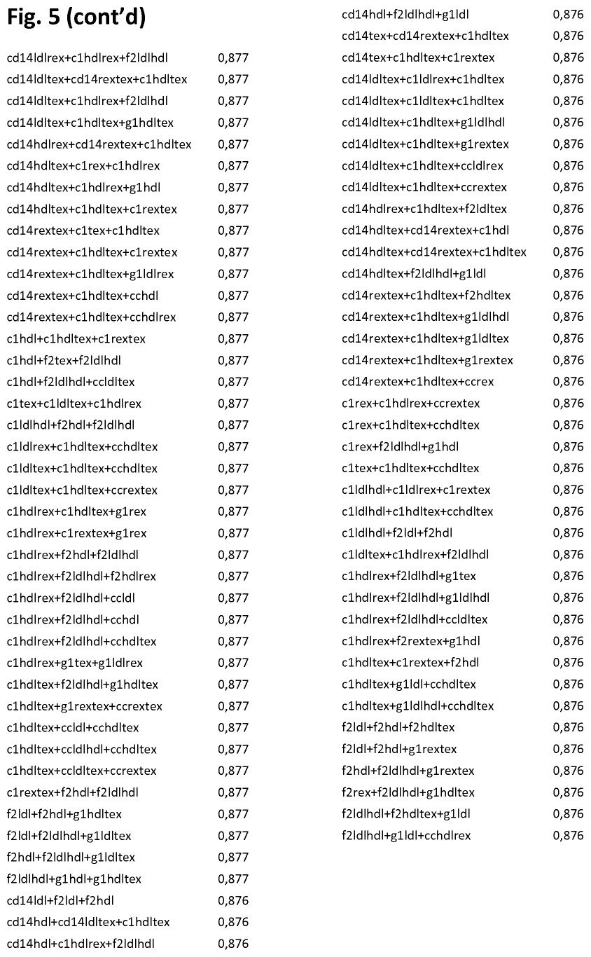

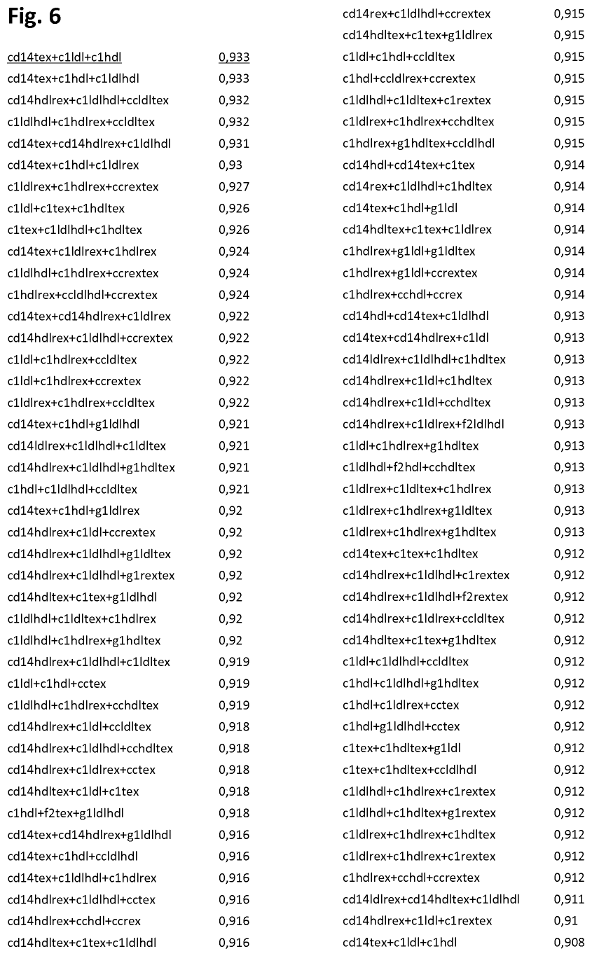

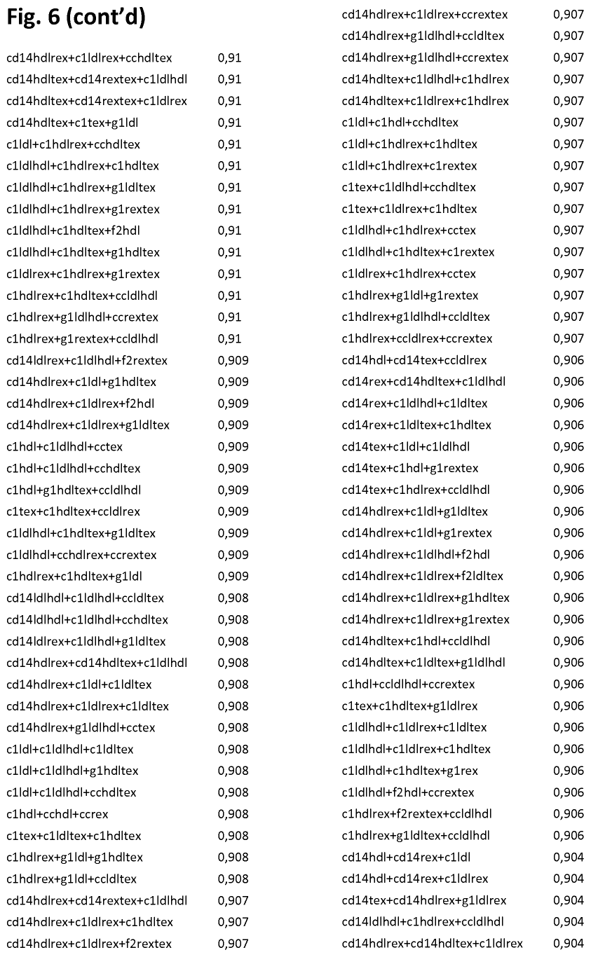

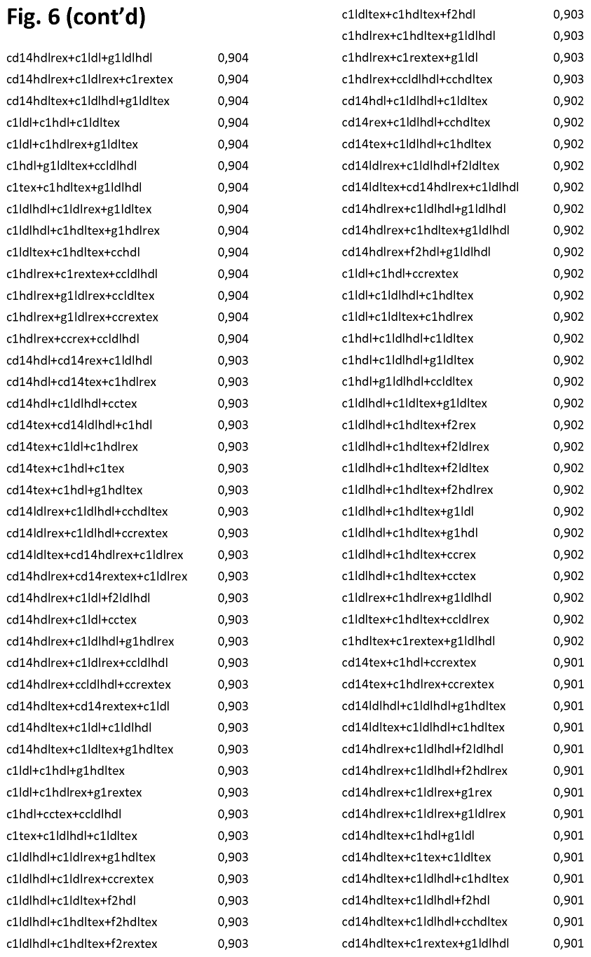

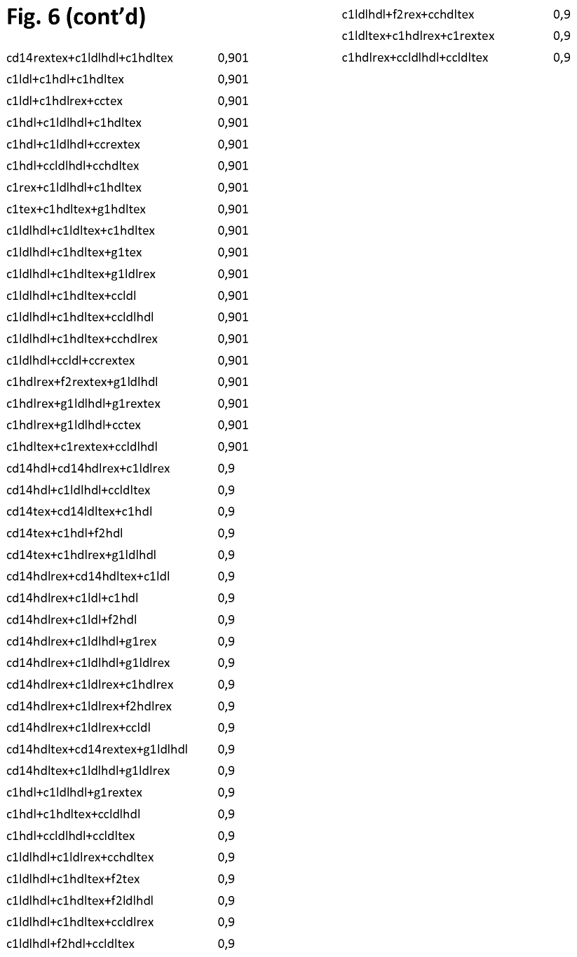

The one or more values of marker/fraction and/or marker/ratio pairs selected in step b), are selected based on their individual or combined statistical relevance for identification of a subject suffering from, or at risk of suffering from an ischemic heart disease such as, unstable and stable angina. The statistical relevance can be determined in any way known to the person skilled in the art. Logistic regression analysis is often used to predict a binary outcome (yes or no). In medical research it is often used to predict if a patient has a certain disease, for example diabetes (yes or no) by modelling observed characteristics of the patients e.g. sex, age, weight and systolic blood pressure. In the diabetes example a result from logistic regression could be that a 10-year increase in age gives a 20% higher odds of having diabetes. By combining more probabilities, based on more than one patient characteristic in one prediction model, one can even more accurately predict if a patient has diabetes or not. Next to using patient characteristics like sex and age, logistic regression can also be performed with biomarker levels associated with extracellular vesicles as potential predictors. A set of biomarkers can be combined in one model and used to estimate the probability of a disease. The performance of a logistic regression model can be visualized in a Receiver Operating Characteristic (ROC) plot. The higher the Area Under the Curve (AUC), the better the performance of the model. An AUC of 0.5 corresponds to a 50% chance of the disease in question (flipping a coin). Here, the inventors used logistic regression and ROC plot analysis in order to evaluate whether the diagnosis of stable coronary artery disease and unstable angina could be improved based on the five extracellular vesicle proteins in 4 different plasma sub-fractions and whether such be better than by random chance. The present inventors determined the statistical relevance of the different individual marker/fraction pairs and marker/ratio pairs (tables 1 and 3) and of combinations thereof (tables 2 and 4 and FIGS. 5 and 6) by ROC plot analysis. The ROC plot analysis involves:
analyzing Differences in baseline-characteristics using Chi-square test for categorical variables
performing T-tests for normally distributed continuous variables and Mann-Whitney-U-tests for continuous variables that were not normally distributed
converting the one or more values derived from the concentration in a given plasma fraction or ratio of concentrations in two different plasma fractions into standard deviation units, or the z-score, by using the observed value minus the mean value, divided by the standard deviation, and
Performing Receiver-operating characteristic (ROC) analysis (ROC plot) to determine the area under the curve (AUC) and optimal calculated cutoff for the negative predictive value (NPV) and/or the positive predictive value (PPV).

This statistical analysis can be performed by a any suitable software, for example by SPSS® (IBM®, Version 22) and Rstudio using R software for statistical computing version 3.1.2. The skilled person will understand, that where more than one value of marker/fraction and/or marker/ratio pairs are used, the AUC and calculated cut-off value obtained from the ROC plot analysis relate to the combination of said values.

In one embodiment of the present invention the one or more values in step b) are selected such that the area under the curve (AUC) differs from the diagonal reference line of 0.5 with a p-value of 0.05 or less, as determined by Receiver Operating Characteristic (ROC) plot analysis of the combination of said one or more values based on a suitable group of definitive subjects and group of reference subjects. In a further embodiment, the one or more values in step b) are selected such that the area under the curve (AUC) is 0.8 or more, such as 0.85 or more, 0.9 or more or 0.95 or more, and the p-value is 0.05 or less, as determined by Receiver Operating Characteristic (ROC) plot analysis of the combination of said one or more values based on a suitable group of definitive subjects and group of reference subjects. In yet a further embodiment, the negative predictive value and/or the positive predictive value is 0.8 or more, such as 0.85 or more, 0.9 or more or 0.95 or more, for the selected one or more values in step b), when using the optimal cut-off value as determined by Receiver Operating Characteristic (ROC) plot analysis of the combination of said one or more values based on a suitable group of definitive subjects and group of reference subjects. In the context of the present invention the term “group of definitive subjects” means a group of subjects that has been verified to suffer from an ischemic heart disease, such as stable angina or unstable angina, by other means than by the method of the present invention. This group could also be called the positive control group. The term “group of reference subjects” as used herein, is a group of subjects that has been verified not to suffer from an ischemic heart disease, such as stable angina or unstable angina, by other means than by the method of the present invention. This group could also be called the negative control group. In one embodiment of the present invention, the group of reference subjects have the same clinical signs and symptoms as the group of definitive cases, but are not suffering from ischemic heart disease. In a further embodiment, the suitable group of definitive subjects and group of reference subjects is a suitable cohort, such as a cohort selected from the group consisting of the Minerva cohort and the Myomarker cohort. In the context of the present invention, the Minerva cohort comprises a group of definitive subjects, which have been diagnosed to suffer from unstable angina and a group of reference subjects, which have the same clinical signs and symptoms as the group of definitive cases, but which are not suffering from unstable angina, wherein the assessment of whether or not a subject falls in the group of definitive cases or in the group of reference subjects is performed by other means than by the method of the present invention. In the context of the present invention, the Myomarker cohort comprises a group of definitive subjects, which have been diagnosed to suffer from stable angina and a group of reference subjects, which have the same clinical signs and symptoms as the group of definitive cases, but which are not suffering from stable angina, wherein the assessment of whether or not a subject falls in the group of definitive cases or in the group of reference subjects is performed by other means than by the method of the present invention. The comparison performed in step c) of the method according to the present invention can therefore alternatively be implemented as performing a comparison using a model and a cut-off value as determined by Receiver Operating Characteristic (ROC) plot analysis of the combination of said one or more values based on a suitable group of definitive subjects and group of reference subjects, wherein the outcome of said model is indicative of the subject suffering from, or being at risk of suffering from, an ischemic heart disease, such as stable angina or unstable angina.

In a further embodiment, the present invention relates to a method for determining one or more values derived from a concentration of a protein marker in a plasma fraction or from a ratio of concentrations of a single protein marker in two different plasma fractions, said method comprising the steps of:
a) obtaining one or more of the plasma fractions selected from the group consisting of the Low-Density Lipoprotein (LDL) fraction, the High-Density Lipoprotein (HDL) fraction, the remaining (REX) fraction and the total (TEX) fraction from a plasma sample of said subject;
b) determining one or more values, wherein each of the one or more values:
   is derived from a concentration of a protein marker in one of said plasma fractions, wherein the protein marker is selected from the group consisting of SerpinC1, Cystatin C, CD14, SerpinF2 and SerpinG1, and/or
   is a ratio of two values derived from the concentrations of a single protein marker in two different ones of said plasma fractions, wherein the protein marker is selected from the group consisting of SerpinC1, Cystatin C, CD14, SerpinF2 and SerpinG1.

The method may further comprise performing a comparison of the one or more values as determined in step b) with one or more corresponding reference values, which has been derived in the same way from the concentration of the same one or more protein marker in corresponding plasma fractions as determined in a group of reference subjects not suffering from ischemic heart disease. When there is a statistically significant difference between the one or more values determined in step b) and the one or more corresponding reference values, it is indicative of the subject suffering, or being at risk of suffering, from an ischemic heart disease.

### Unstable Angina

The inventors of the present invention showed earlier that the protein content of all plasma extracellular vesicles at 6 hours after the onset of symptoms appeared to be associated with the diagnosis of non ST-elevation myocardial infarction (De Hoog et al.

Serum extracellular vesicle protein levels are associated with acute coronary syndrome. Eur Heart J Acute Cardiovasc Care 2013. 2(1):53-60) establishing that the content of unfractionated (total) plasma extracellular vesicles is rapidly changing after ischemic heart disease. In the current invention, a different sample of plasma sub-fractions was used, in which the inventors assessed the extracellular vesicle protein content. Plasma samples were derived from patients with chest pain at the emergency department that were finally diagnosed to suffer from unstable angina and from individuals that also entered the emergency department with chest pain but that did not have ischemic heart disease (serving as controls). The inventors performed proteomics on the extracellular vesicle sub-fractions from both groups. Proteins were characterized in the extracellular vesicles present in the LDL, HDL, REX and TEX extracellular plasma sub-fractions of in total 30 established unstable angina chest pain patients and in a total of 30 of the age-, sex-, risk-, history- and medication-matched control chest pain emergency department patients (without unstable angina).

In one embodiment, the present invention relates to a method for identifying a subject suffering from, or being at risk of suffering from, unstable angina, said method comprising the steps of:
a) obtaining one or more of the plasma fractions selected from the group consisting of the Low-Density Lipoprotein (LDL) fraction, the High-Density Lipoprotein (HDL) fraction, the remaining (REX) fraction and the total (TEX) fraction from a plasma sample of said subject;
c) determining one or more values selected from the group consisting of values derived from the concentration of:
   SerpinC1 in the HDL plasma fraction,
   CD14 in the HDL plasma fraction,
   CD14 in the REX plasma fraction,
   CD14 in the TEX plasma fraction, and
   SerpinF2 in the TEX plasma fraction,
and/or wherein at least one of the one or more values is a ratio of two values derived from concentrations of a single protein marker in two different ones of said plasma fractions, selected from the group consisting of:
   the concentration of SerpinC1 in the LDL plasma fraction over the concentration in the HDL plasma fraction,
   the concentration of SerpinC1 in the HDL plasma fraction over the concentration in the REX plasma fraction,
   the concentration of SerpinC1 in the HDL plasma fraction over the concentration in the TEX plasma fraction,
   the concentration of CD14 in the LDL plasma fraction over the concentration in the HDL plasma fraction,
   the concentration of CD14 in the LDL plasma fraction over the concentration in the TEX plasma fraction,
   the concentration of CD14 in the HDL plasma fraction over the concentration in the REX plasma fraction,
   the concentration of CD14 in the HDL plasma fraction over the concentration in the TEX plasma fraction,
   the concentration of Cystatin C in the LDL plasma fraction over the concentration in the HDL plasma fraction,
   the concentration of Cystatin C in the HDL plasma fraction over the concentration in the REX plasma fraction,
   the concentration of Cystatin C in the HDL plasma fraction over the concentration in the TEX plasma fraction, and
   the concentration of SerpinF2 in the REX plasma fraction over the concentration in the TEX plasma fraction, and
c) performing a comparison of the one or more values as determined in step b) with one or more corresponding reference values, which has been derived in the same way from the concentration of the same one or more protein marker in corresponding plasma fractions as determined in a group of reference subjects not suffering from unstable angina, wherein a statistically significant difference between the one or more values determined in step b) and the one or more corresponding reference values is indicative of the subject suffering, or being at risk of suffering, from unstable angina.

The present inventors have found that these marker/fraction and marker/ratio values provide a statistically relevant identification of subjects suffering from unstable angina on their own (see table 1). Accordingly, combination with further values of marker/fraction pairs and/or marker/ratio pairs is not required but will in many cases improve the certainty by which a subject is correctly identified as suffering from unstable angina. Accordingly, the method according to this embodiment of the invention provides for the first time a method to identify subjects suffering from unstable angina from a blood sample with a statistical relevance characterized by that the one or more values in step b) are selected such that the area under the curve (AUC) differs from the diagonal reference line of 0.5 with a p-value of 0.05 or less, as determined by Receiver Operating Characteristic (ROC) plot analysis of the combination of said one or more values based on a suitable group of definitive subjects and group of reference subjects. Preferably, the statistical relevance of the method according to the invention is characterized by that the one or more values in step b) are selected such that the area under the curve (AUC) is 0.8 or more, such as 0.85 or more, 0.9 or more or 0.95 or more and the p-value is 0.05 or less, as determined by Receiver Operating Characteristic (ROC) plot analysis of the combination of said one or more values based on a suitable group of definitive subjects and group of reference subjects. Even more preferably, the statistical relevance of the method according to the invention is characterized by resulting in a negative predictive value and/or a positive predictive value is 0.8 or more, such as 0.85 or more, 0.9 or more or 0.95 or more.

The present inventors have found that the marker/fraction value of SerpinC1 in the HDL and the marker/ratio values of SerpinC1 in LDL/HDL, SerpinC1 in HDL/REX, SerpinC1 in HDL/TEX, CD14 in LDL/HDL, CD14 in HDL/REX and CD14 in HDL/TEX all individually provide an AUC value of 0.8 or more and a p-value of 0.05 or less in a ROC plot performed as described above. Accordingly, combination with further values of marker/fraction pairs and/or marker/ratio pairs is not required, but will in many cases improve the certainty by which a subject is correctly identified as suffering from unstable angina. A preferred embodiment of the invention is therefore a method for identifying a subject suffering from, or being at risk of suffering from, unstable angina, at least one of the one or more values determined in step b) is derived from the concentration of SerpinC1 in the HDL plasma fraction and/or at least one of the one or more values is a ratio of two values derived from concentrations of a single protein marker in two different ones of said plasma fractions, selected from the group consisting of:
the concentration of SerpinC1 in the LDL plasma fraction over the concentration in the HDL plasma fraction,
the concentration of SerpinC1 in the HDL plasma fraction over the concentration in the REX plasma fraction,
the concentration of SerpinC1 in the HDL plasma fraction over the concentration in the TEX plasma fraction,
the concentration of CD14 in the LDL plasma fraction over the concentration in the HDL plasma fraction,
the concentration of CD14 in the HDL plasma fraction over the concentration in the REX plasma fraction, and
the concentration of CD14 in the HDL plasma fraction over the concentration in the TEX plasma fraction.

Tables 1 and 3 in example 4 show the marker/fraction pairs and marker/ratio pairs which provide the most statistically relevant identification of unstable and stable angina, respectively. By comparison of tables 1 and 3 it follows that the values of marker/ratio pairs SerpinC1-LDL/HDL, CD14-LDL/HDL and CD14-HDL/REX, have AUC values of 0.8 or more for identification of unstable angina on their own (without combination with a further marker/fraction or marker/ratio value). Although these marker/ratios are also significant for identification of stable angina, their AUC values for stable angina are lower than 0.8, and these marker/ratios are therefore more significant for unstable angina, than for stable angina. It also follows that the marker/ratio pairs CD14-LDL/TEX and F2-REX/TEX and the marker/fraction pairs CD14-REX, CD14-TEX and F2-TEX are shown in table 1 as being among the most significant single marker/fraction and marker/ratio pairs for identification of unstable angina, whereas they are not among the most significant single marker/fraction and marker/ratio pairs listed for identification of stable angina in table 3. Accordingly, in preferred embodiment of the method for identifying a subject suffering from, or being at risk of suffering from, unstable angina, at least one of the one or more values is selected from the group consisting of values derived from the concentration of:
CD14 in the REX plasma fraction,
CD14 in the TEX plasma fraction, and
SerpinF2 in the TEX plasma fraction,
   and/or wherein at least one of the one or more values is a ratio of two values derived from concentrations of a single protein marker in two different ones of said plasma fractions, selected from the group consisting of:
the concentration of SerpinC1 in the LDL plasma fraction over the concentration in the HDL plasma fraction,
the concentration of CD14 in the LDL plasma fraction over the concentration in the HDL plasma fraction,
the concentration of CD14 in the LDL plasma fraction over the concentration in the TEX plasma fraction,
the concentration of CD14 in the HDL plasma fraction over the concentration in the REX plasma fraction, and
the concentration of SerpinF2 in the REX plasma fraction over the concentration in the TEX plasma fraction.

In another preferred embodiment of the method for identifying a subject suffering from, or being at risk of suffering from, unstable angina, at least one of the one or more values is selected from the group consisting of values derived from the concentration of:
CD14 in the REX plasma fraction,
CD14 in the TEX plasma fraction, and
SerpinF2 in the TEX plasma fraction,
   and/or wherein at least one of the one or more values is a ratio of two values derived from concentrations of a single protein marker in two different ones of said plasma fractions, selected from the group consisting of:
the concentration of CD14 in the LDL plasma fraction over the concentration in the TEX plasma fraction,
the concentration of SerpinF2 in the REX plasma fraction over the concentration in the TEX plasma fraction.

As outlined in more detail in table 1 in example, 4 the present invention provides a number of individual markers that outperformed other proteins: For example, SerpinC1 with the ratio HDL/REX and an AUC of 0.855 (95% Cl 0.753-0.957); CD14 with the ratio HDL/REX and an AUC of 0.847 (95% Cl 0.746-0.948) and SerpinC1 in the HDL fraction with an AUC of 0.844 (95% Cl 0.741-0.947) to discriminate between unstable angina and matched controls.

### Stable Angina

Although unstable angina is thought to be a thrombotic event in contrast to stable angina, there is mechanistic overlap between the two clinical syndromes: there is coronary artery disease and myocardial ischemia (reduction in blood flow) in both disorders. For this, the inventors performed similar immuno bead assay experiments on blood (plasma) samples from 30 established stable coronary artery disease chest pain patients versus 30 age-, sex-, risk-, history-, and medication-matched control chest pain patients in the extracellular plasma sub-fractions LDL, HDL, REX and TEX.

In a one embodiment, the present invention relates to a method for identifying a subject suffering from, or being at risk of suffering from, stable angina, said method comprising the steps of:
a) obtaining one or more of the plasma fractions selected from the group consisting of the Low-Density Lipoprotein (LDL) fraction, the High-Density Lipoprotein (HDL) fraction, the remaining (REX) fraction and the total (TEX) fraction from a plasma sample of said subject;
b) determining at least one of the one or more values is selected from the group consisting of values derived from the concentration of:
   SerpinC1 in the HDL plasma fraction,
   SerpinC1 in the TEX plasma fraction, and
   CD14 in the HDL plasma fraction,
and/or wherein at least one of the one or more values is a ratio of two values derived from concentrations of a single protein marker in two different ones of said plasma fractions, selected from the group consisting of:
   the concentration of SerpinC1 in the LDL plasma fraction over the concentration in the HDL plasma fraction,
   the concentration of SerpinC1 in the HDL plasma fraction over the concentration in the REX plasma fraction,
   the concentration of SerpinC1 in the HDL plasma fraction over the concentration in the TEX plasma fraction,
   the concentration of SerpinC1 in the REX plasma fraction over the concentration in the TEX plasma fraction,
   the concentration of CD14 in the LDL plasma fraction over the concentration in the HDL plasma fraction,
   the concentration of CD14 in the HDL plasma fraction over the concentration in the REX plasma fraction,
   the concentration of CD14 in the HDL plasma fraction over the concentration in the TEX plasma fraction,
   the concentration of Cystatin C in the LDL plasma fraction over the concentration in the HDL plasma fraction,
   the concentration of Cystatin C in the HDL plasma fraction over the concentration in the REX plasma fraction,
   the concentration of Cystatin C in the HDL plasma fraction over the concentration in the TEX plasma fraction,
   the concentration of SerpinF2 in the LDL plasma fraction over the concentration in the HDL plasma fraction,
   the concentration of SerpinF2 in the LDL plasma fraction over the concentration in the REX plasma fraction,
   the concentration of SerpinF2 in the HDL plasma fraction over the concentration in the TEX plasma fraction, and
   the concentration of SerpinG1 in the REX plasma fraction over the concentration in the TEX plasma fraction, and
c) performing a comparison of the one or more values as determined in step b) with one or more corresponding reference values, which has been derived in the same way from the concentration of the same one or more protein marker in corresponding plasma fractions as determined in a group of reference subjects not suffering from stable angina, wherein a statistically significant difference between the one or more values determined in step b) and the one or more corresponding reference values is indicative of the subject suffering, or being at risk of suffering, from stable angina.

The present inventors have found that these values of marker/fraction pairs and marker/ratio pairs provide a statistically relevant identification of subjects suffering from stable angina on their own (see table 3). Accordingly, combination with further marker/fraction and/or marker/ratio values is not required but will in many cases improve the certainty by which a subject is correctly identified as suffering from stable angina. Accordingly, the method according to this embodiment of the invention provides for the first time a method to identify subjects suffering from stable angina from a blood sample with a statistical relevance characterized by that the one or more values in step b) are selected such that the area under the curve (AUC) differs from the diagonal reference line of 0.5 with a p-value of 0.05 or less, as determined by Receiver Operating Characteristic (ROC) plot analysis of the combination of said one or more values based on a suitable group of definitive subjects and group of reference subjects. Preferably, the statistical relevance of the method provided in this embodiment of the invention is characterized by that the one or more values in step b) are selected such that the area under the curve (AUC) is 0.8 or more, such as 0.85 or more, 0.9 or more or 0.95 or more and the p-value is 0.05 or less, as determined by Receiver Operating Characteristic (ROC) plot analysis of the combination of said one or more values based on a suitable group of definitive subjects and group of reference subjects. Even more preferably, the statistical relevance of this embodiment of the invention is characterized by resulting in a negative predictive value and/or a positive predictive value is 0.8 or more, such as 0.85 or more, 0.9 or more or 0.95 or more.

The present inventors have found that the marker/fraction value of SerpinC1 in the HDL and the marker/ratio values of SerpinC1 in HDL/REX, SerpinC1 in HDL/TEX, CD14 in HDL/TEX and SerpinF2 in LDL/HDL all individually provide an AUC value of 0.8 or more and a p-value of 0.05 or less in a ROC plot performed as described above. Accordingly, combination with further marker/fraction and/or marker/ratio values is not required, but will in many cases improve the certainty by which a subject is correctly identified as suffering from stable angina. A preferred embodiment of the present invention is therefore a method for identifying a subject suffering from, or being at risk of suffering from, stable angina, at least one of the one or more values is derived from the concentration of SerpinC1 in the HDL plasma fraction and/or at least one of the one or more values is a ratio of two values derived from concentrations of a single protein marker in two different ones of said plasma fractions, selected from the group consisting of:
the concentration of SerpinC1 in the HDL plasma fraction over the concentration in the REX plasma fraction,
the concentration of SerpinC1 in the HDL plasma fraction over the concentration in the TEX plasma fraction,
the concentration of CD14 in the HDL plasma fraction over the concentration in the TEX plasma fraction,
the concentration of SerpinF2 in the LDL plasma fraction over the concentration in the HDL plasma fraction.

Tables 1 and 3 in example 4 show the marker/fraction and marker/ratio pairs which give the most statistically relevant identification of unstable and stable angina, respectively. By comparison of tables 1 and 3 it follows that the F2-LDL/HDL marker/ratio pair has an AUC value of above 0.8 for identification of stable angina on its own (without combination with a further marker/fraction or marker/ratio value), whereas it is not listed in table 1 as one of the most significant marker/ratio pairs for identification of unstable angina. It also follows that the marker/ratio pairs C1-REX/TEX, F2-LDL/REX, F2-HDL/TEX and G1-REX/TEX and the C1-TEX marker/fraction pair are shown in table 3 as being among the most significant single marker/fraction and marker/ratio pairs for identification of stable angina, whereas they are not among the most significant marker/fraction and marker/ratio pairs listed for identification of unstable angina in table 1. Accordingly, in a preferred embodiment of the method for identifying a subject suffering from, or being at risk of suffering from, stable angina, at least one of the one or more values is selected from the group consisting of values derived from the concentration of SerpinC1 in the TEX plasma fraction and/or wherein at least one of the one or more values is a ratio of two values derived from concentrations of a single protein marker in two different ones of said plasma fractions, selected from the group consisting of:
the concentration of SerpinC1 in the REX plasma fraction over the concentration in the TEX plasma fraction,
the concentration of SerpinF2 in the LDL plasma fraction over the concentration in the HDL plasma fraction,
the concentration of SerpinF2 in the LDL plasma fraction over the concentration in the REX plasma fraction,
the concentration of SerpinF2 in the HDL plasma fraction over the concentration in the TEX plasma fraction, and
the concentration of SerpinG1 in the REX plasma fraction over the concentration in the TEX plasma fraction.
   Combination of Marker/Fraction and/or Marker/Ratio Pairs.

### Unstable Angina

Table 2 in example 4 shows the statistically most relevant combinations of two marker/fraction and/or marker/ratio pairs for the identification of unstable angina in the Minerva cohort. It follows from table 2, that the statistically most significant combination of two marker/fractions and/or marker/ratio pairs for identification of unstable angina as determined in herein is the combination of SerpinC1-LDL/HDL and SerpinC1-HDL/TEX with an AUC of 0.902 as determined by ROC plot analysis as described in the above. With an optimal cut-off value based on the AUC, this combination of SerpinC1-LDL/HDL and SerpinC1-HDL/TEX has a sensitivity of 0.851 (95% Cl 0.663-0.958), a specificity of 0.839 (95% Cl 0.663-0.945), a negative predictive value (NPV) of 0.867 (95% Cl 0.690-0956) and a positive predictive value of 0.821 (95% Cl 0.635-0.948). The table in FIG. 6 shows the statistically most relevant combinations of three of marker/fraction and/or marker/ratio pairs in the Minerva cohort. It follows from the table in FIG. 6, that the statistically most significant combination of three marker/fractions and/or marker/ratio pairs for identification of unstable angina as determined in herein is the combination of SerpinC1-HDL+CD14-TEX+SerpinC1-LDL showing an AUC of 0.933 as determined by ROC plot analysis as described in the above. With an optimal cut-off value based on the AUC, the sensitivity of these three markers was 92.6% and specificity of 87%. The Negative Predictive Value (NPV) is 93.1% and the Positive Predictive Value is 86.2%. The combination of SerpinC1-HDL+CD14-TEX+SerpinC1-LDL was thus concluded to be one of the more the optimal combinations (panels) for the identification of a subject suffering from unstable angina. This means that determining the levels of SerpinC1 in LDL, HDL and REX, and CD14 in HDL and TEX is one of the more optimal ways of diagnosing unstable angina very accurately, while only a blood sample of less than 250 μl is required. With an NPV of 93.1% it can also determine which patients can be send home safely. Hence, the inventors of the present invention have now found the method and means to discriminate between patients that experience unstable angina and should be further treated, from patients that also have chest pain, but that do not suffer from unstable angina, by preferably applying a protein concentration determination of SerpinC1 in LDL, HDL and REX, and CD14 in HDL and TEX, and more preferably by applying a protein concentration determination of SerpinC1 in HDL and LDL, and CD14 in TEX, and comparing the concentrations with those found in a control sample. This now enables one to perform a rapid and reliable diagnostic test based on a single (and small) blood sample.

### Stable Angina

Table 4 in example 4 shows the statistically most relevant combinations of two marker/fraction and/or marker/ratio pairs for the identification of stable angina in the Myomarker cohort. It follows from table 4, that the statistically most significant combination of two marker/fractions and/or marker/ratio pairs for identification of stable angina as determined in herein is the combination of SerpinC1-HDL and SerpinF2-LDL/HDL with an AUC of 0.881 as determined by ROC plot analysis as described in the above. With an optimal cut-off value based on the AUC, this combination of SerpinC1-HDL and SerpinF2-LDL/HDL has a sensitivity of 0.786 (95% Cl 0.590-0.917), a specificity of 0.852 (95% Cl 0.633-0.958), a negative predictive value (NPV) of 0.793 (95% Cl 0.601-0.938) and a positive predictive value of 0.846 (95% Cl 0.652-0.943). The table in FIG. 5 shows the statistically most relevant combinations of three of marker/fraction and/or marker/ratio pairs in the Myomarker cohort. It follows from the table in FIG. 5, that the statistically most significant combination of three marker/fractions and/or marker/ratio pairs for identification of stable angina as determined in herein is the combination of SerpinC1-HDL+SerpinF2-LDL/HDL+SerpinG1-HDL with an AUC of 0.922 as determined by ROC plot analysis as described in the above. With an optimal cut-off value based on the AUC, the sensitivity of these three markers was 0.857 (95% Cl 0.673-0.960) and specificity of 0.852 (95% Cl 0.663-0.958). The Negative Predictive Value (NPV) was 0.852 (95% Cl 0.664-0.958) and the Positive Predictive Value was 0.857 (95% Cl 0.672-0.960). The present inventors found that SerpinC1 is one of the better protein markers when using only one marker/fraction or marker/ratio pair. From table 3 it follows that the 3 best individual marker/fraction or marker ratio pairs are SerpinC1 with the ratio HDL/TEX and an AUC of 0.842 (95% Cl 0.741-0.944); SerpinC1 with the ratio HDUREX and an AUC of 0.828 (95% Cl 0.718-0.938)) and SerpinC1 in the HDL fraction with an AUC of 0.812 (95% Cl 0.700-0.924) to discriminate between stable angina and matched controls. Selection of the best combination of markers and sub-fractions without the ratios of markers between the sub-fractions showed an AUC of 0.861, when not taking any marker/ratio pair into account. It was concluded that one of the more optimal combination (panel) to diagnose stable angina appeared to consist of SerpinC1-HDL+SerpinG1-TEX+SerpinC1-REX. With an optimal cut-off value based on the AUC, the sensitivity of these 3 markers was 93.3% and specificity of 76.7%. The NPV is 92% and the Positive Predictive Value is 80%. Hence, the inventors of the present invention have now also found a method and means to discriminate between patients that experience stable angina and should be further treated, from patients that also have chest pain, but that do not suffer from stable angina, by preferably applying a protein concentration determination of SerpinC1 in HDL, REX and TEX, and more preferably by applying a protein concentration determination of SerpinC1 in HDL and REX, and SerpinG1 in TEX, and comparing the concentrations with those found in a control sample. Although applying a different cohort and patient group, it is concluded here also that a similar blood-based assay as outlined above for unstable angina, can now determine whether patients suffer from stable angina or not.

In conclusion, the skilled person will understand, that whereas the marker/fraction and marker/ratio pairs identified as having a statistically significant predictive value for the identification of subjects suffering from, or at risk of suffering from, an ischemic heart disease, such as stable or unstable angina, the predictive value will increase if more than one, such as at least two or at least three of the identified marker/fraction or marker/ratio pairs are included in the methods according to the present invention. In one embodiment of the present invention, the method for identifying a subject suffering from, or being at risk of suffering from, an ischemic heart disease, such as stable or unstable angina, comprises that at least two values, such as at least three values, are determined in step b). In another embodiment of the present invention, the method for identifying a subject suffering from, or being at risk of suffering from, an ischemic heart disease, such as stable or unstable angina, comprises that at least one of the one or more values determined in step b) is a ratio of two values derived from concentrations of a single protein marker in two different ones of said plasma fractions, wherein the protein marker is selected from the group consisting of SerpinC1, Cystatin C, CD14, SerpinF2 and SerpinG1. In another embodiment at least two, such as at least three, values are determined in step b) and at least one of these values is a ratio of two values derived from concentrations of a single protein marker in two different ones of said plasma fractions. In another embodiment of the present invention, at least two values involving at least two different protein markers are determined in step b), which values each can be selected from a value of a marker/fraction pair or a marker/ratio pair. In yet a further embodiment of the present invention at least one of said protein markers is SerpinC1, meaning that either a marker/fraction pair or a marker/ratio pair involving SerpinC1 is used in the method for identifying a subject suffering from, or being at risk of suffering from, an ischemic heart disease, such as stable or unstable angina. When one of the protein markers used in the method according to the present invention is SerpinC1, and at least one further protein marker is used in combination herewith, said at least one further protein marker is suitably selected from the group consisting of Cystatin C, CD14, SerpinF2 and SerpinG1. Suitably, all selected protein markers are different.

### Kit

The present invention also relates to a kit comprising means for performing the method according to the invention. In one embodiment, the kit according to the invention comprises means for determining the concentration of said protein markers in said fractions. Said means may comprise any suitable means known in the art, such as an immune-bead based process as outlined herein. Further, the kit may comprise instructions and means for the fractionation of a plasma sample to enable the rapid fractionation and subsequent protein concentration determination as outlined herein. The kit may also comprise means for performing the comparison of step c) in the form of an algorithm with a suitable cut-off value. Preferably, the cut-off value is determined by Receiver Operating Characteristic (ROC) plot analysis of the combination of said one or more values based on a suitable group of definitive subjects and group of reference subjects.

Patients with suspected ischemic heart disease constitute an enormous health care burden. Rapid and straightforward diagnostic tools for stable and unstable angina are currently not available. The technology used in this invention to characterize the protein content of the extracellular vesicles of the different plasma sub-fractions and the subsequent immune-based detection of the indicated proteins in such fractions is suitable for automated settings and ideal for point of care applications, and has major implications for the efficiency of the diagnostic process and clinical management. The diagnosis of stable and unstable angina with the test as indicated herein now enables timely and adequate subsequent treatment. It will have a significant impact on the prognosis of the patient. On the other hand, ruling out both diseases prevents unnecessary hospital referrals/admissions that are expensive, time consuming and sometimes contain risky additional testing.

### Further Embodiments

The present invention relates to a method for diagnosing a human patient as suffering from ischemic heart disease, said method comprising: obtaining a Low-Density Lipoprotein (LDL), a High-Density Lipoprotein (HDL), a remaining (REX) and a total (TEX) fraction of a plasma sample from said patient; determining the concentration of a protein marker in said LDL, HDL, REX and TEX fractions; determining the concentration of said protein marker in reference fractions from a human subject not suffering from ischemic heart disease; and determining whether said patient suffers from ischemic heart disease based on the difference in the concentrations determined in the fractions of said patient and said subject not suffering from ischemic heart disease, wherein said protein marker is selected from the group consisting of: human SerpinC1, SerpinG1, CD14 and SerpinF2. Preferably, the concentration of SerpinC1 is determined. In another aspect of the invention the concentration of SerpinC1 and CD14 are measured and combined to determine whether a patient suffers from unstable angina. In another aspect of the invention the concentration of SerpinC1 and SerpinG1 are measured and combined to determine whether a patient suffers from stable angina. The inventors of the present invention have found that particular proteins are differentially expressed between sub-fractions of plasma samples of human patients with ischemic heart disease and reference sub-fractions of plasma samples from human subjects that do not suffer from ischemic heart disease. Reference samples means that the plasma samples are treated in the same manner and sub-fractions LDL, HDL, REX and TEX are compared. Preferably, to have a proper comparison between patient and non-patient (yes/no suffering from ischemic heart disease) said human patient and said human subject not suffering from ischemic heart disease both experience chest pain, indicative of ischemic heart disease. However, the human subject not suffering from ischemic heart disease may have entered the emergency room with chest pain that appeared indicative of ischemic heart disease. Of course the skilled person would understand that other reference samples may also be used in the comparison, for instance those that are obtained from human subjects that are healthy and did not experience chest pain or other symptoms that are related, or that are indicative of ischemic heart disease. In a highly preferred embodiment, said ischemic heart disease is stable angina or unstable angina. As outlined above, no rapid and reliable protein-based markers are available in the art that enable a physician or GP to determine quickly whether a human patient suffering from symptoms that are indicative of stable or unstable angina, do indeed suffer from stable or unstable angina. Such is now presented herein: A plasma sample from a suspected ischemic heart disease patient is fractionated into sub-fractions and the concentration of a single or a set of proteins is determined in said fractions. It is known that the proteins in said fractions are associated with the extracellular vesicles within said fractions. Subsequently, the concentrations are compared to concentrations determined in reference samples from a control human subject and when compared, the determined concentration in said fractions can now tell the physician or GP whether a suspected patient indeed suffers from ischemic heart disease, preferable stable or unstable angina.

In a particularly preferred aspect of the invention, said ischemic heart disease is unstable angina, and for this the concentrations of SerpinC1 and CD14 are determined. More preferably in the case of unstable angina, the concentration of SerpinC1 is determined in the LDL, HDL and/or REX fractions and the concentration of CD14 is determined in the HDL, REX and/or TEX fractions. As outlined in the accompanying examples, the inventors found that especially the combination of SerpinC1-HDL+CD14-TEX+SerpinC1-LDL was indicative for unstable angina. In another preferred aspect, SerpinC1 in HDL/REX appeared indicative, CD14 in HDL/REX appeared indicative and SerpinC1 in HDL alone appeared indicative.

In another particularly preferred aspect of the invention, said ischemic heart disease is stable angina, and for this the concentrations of SerpinC1 and SerpinG1 are determined. More preferably in the case of stable angina, the concentration of SerpinC1 is determined in the HDL, REX and/or TEX fractions, and the concentration of SerpinG1 is determined in the TEX fraction. As outlined in the accompanying examples, the inventors found that especially the combination of SerpinC1-HDL+SerpinG1-TEX+SerpinC1-REX was indicative for stable angina. In another preferred aspect, SerpinC1 in HDL/TEX, or HDL/REX or HDL alone appeared indicative.

The present invention also relates to a kit for performing the method according to the invention, said kit comprising the means for determining the concentration of said protein markers in said fractions. Said means may comprise any suitable means known in the art, such as an immune-bead based process as outlined herein. Further, the kit may comprise instructions and means for the fractionation of a plasma sample to enable the rapid fractionation and subsequent protein concentration determination as outlined herein.

The present invention also relates to a protein marker for the diagnosis of stable angina or unstable angina in a human patient, wherein said protein marker is selected from the group consisting of: human SerpinC1, SerpinG1, CD14 and SerpinF2; or wherein said protein marker is a combination of SerpinC1, SerpinG1, CD14 and SerpinF2. The best performing protein marker in the hands of the inventors appeared to be SerpinC1 that appeared indicative for both stable and unstable angina in certain plasma sub-fractions (as outlined in detail herein). The AUC value increased when combinations of protein marker concentrations and certain sub-fractions were combined. For unstable angina it appeared that the best score was obtained when SerpinC1 was determined in HDL and LDL in combination with the concentration of CD14 in TEX (of course compared to their respective values in reference, control samples/fractions). For stable angina it appeared that the best score was obtained when SerpinC1 was determined in HDL and REX in combination with the concentration of SerpinG1 in TEX (of course compared to their respective values in reference, control samples/fractions). SerpinC1 alone in HDL/REX also appeared to have high diagnostic value for unstable angina, while SerpinC1 alone in HDL/TEX appeared to have high diagnostic value for stable angina. Hence, the most preferred biomarker for use in most if not all methods of the present invention is human SerpinC1, when its concentration is determined in LDL, HDL, REX and TEX fractions of a plasma sample from a human patient suspected to suffer from stable or unstable angina.

In yet another embodiment, the present invention relates to a method for determining the level of human SerpinC1, SerpinG1, CD14 and/or SerpinF2 in a plasma sample from a human patient experiencing chest pain, said method comprising: obtaining a plasma sample from said patient; fractionating LDL, HDL, REX and TEX fractions from said plasma sample; and determining the level of human SerpinC1, SerpinG1, CD14 and/or SerpinF2 in the extracellular vesicles present in said fractions. Preferred protein markers are SerpinC1, CD14 and SerpinG1. In respect to unstable angina, highly preferred is a combination of SerpinC1 and CD14, and more preferably the concentration of SerpinC1 is determined in HDL, REX and LDL and the concentration of CD14 is determined in HDL, REX and TEX. In respect of stable angina, highly preferred is a combination of SerpinC1 and SerpinG1, and more preferably the concentration is determined in HDL, TEX and REX and the concentration of SerpinG1 is determined in TEX.

It should be noted that the above-mentioned embodiments illustrate rather than limit the invention, and that those skilled in the art will be able to design many alternative embodiments.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The mere fact that certain measures are recited in mutually different dependent claims does not indicate that a combination of these measures cannot be used to advantage.

### EXAMPLES

### Example 1. Proteomic Study on Minerva Unstable Angina Cohort

The MINERVA study (abbreviation for determination of Microvesicle content IN the Emergency Room: diagnostic Value for Acute coronary syndromes) is a single center, prospective cohort study of patients presenting to the emergency department within 24 hours of the onset of chest pain suggestive of acute coronary syndrome (ACS). Between January 2012 and June 2014, over 2000 consecutive patients were enrolled in the Meander Medical Center Amersfoort, the Netherlands, which is a large regional teaching hospital providing healthcare for a population of ˜300,000 patients. Patients younger than 18 years, patients who were unable or unwilling to give their informed consent and patients with a clear-cut ST-segment elevation MI (STEMI) were not included in this study. All patients were evaluated and managed according to the international guidelines. Along with the routine clinical laboratory measurements directly upon presentation, additional 3×10 cc tubes of venous blood were drawn. The plasma component was frozen and stored at −80° C. within one hour after sample collection.

### Data Acquisition

Detailed patient information was collected and documented in a digital case record form: clinical presentation, medical history, cardiovascular risk factors, current medication use, findings on physical examination, ECG evaluation, blood biochemical parameters and results of additional investigations.

### Diagnosis Adjudication

The primary outcome is ACS (i.e. unstable angina, non ST-segment elevation myocardial infarction (NSTEMI) and ST-segment elevation myocardial infarction (STEMI)), adjudicated according to the universal definition of myocardial infarction and the latest ESC guidelines (Hamm et al. ESC Committee for Practice Guidelines. ESC Guidelines for the management of acute coronary syndromes in patients presenting without persistent ST-segment elevation: The Task Force for the management of acute coronary syndromes (ACS) in patients presenting without persistent ST-segment elevation of the European Society of Cardiology (ESC). Eur Heart J 2011 December; 32(23):2999-3054). The diagnosis of NSTEMI was made when a rise and/or fall in cardiac troponin with at least one value above the 99^{th} percentile was observed in a clinical setting consistent with myocardial ischemia. Unstable angina was diagnosed on the basis of signs and symptoms consistent with myocardial ischemia, accompanied by dynamic ECG changes, evidence of ischemia on functional testing or new coronary angiographic changes. An outcome panel consisting of two cardiologists adjudicated the final diagnosis. In case of uncertainty, a third cardiologist has decided.

### Follow-Up

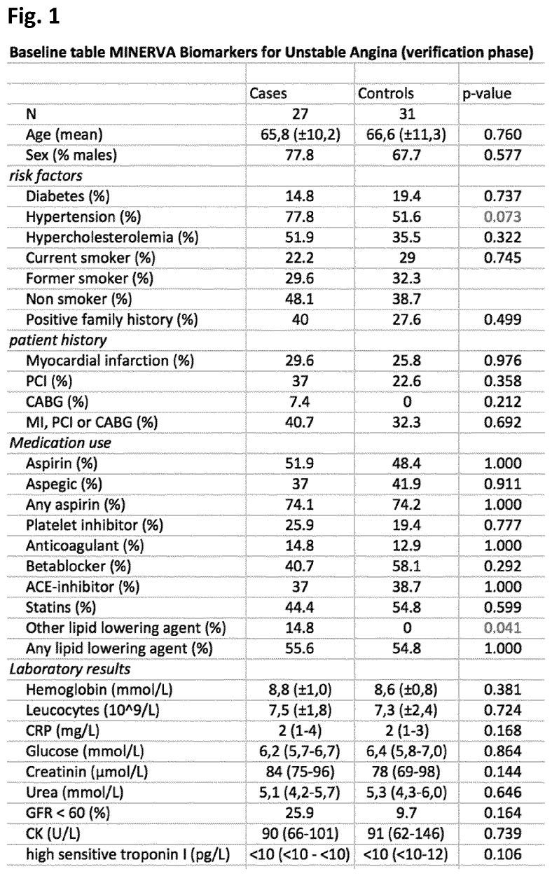

Patients were followed up for at least 1 year after inclusion. Local hospital records were checked for hospitalization (heart failure or unstable angina), myocardial infarction, coronary revascularization and death. Patients were contacted by letter and/or telephone and were asked if they had been admitted to a hospital since inclusion. When this was the case, the hospital records were checked for event verification, also when patients were admitted to a center other than the center of inclusion. Major adverse cardiovascular events were scored, defined as non-fatal MI (STEMI/NSTEMI type 1), coronary revascularization (PCI or CABG), hospital admission for heart failure or unstable angina and/or all cause death during 1 year of follow-up. Verification of the Mass spectrometry discovery was done on 27 definitive UA and 31 controls (see FIG. 1).

### Preparation of Plasma Fractions

Extracellular vesicle plasma sub-fraction isolation using sequential precipitation was generally performed as follows. As previously described (Burnstein et al. Rapid method for the isolation of lipoproteins from human serum by precipitation with polyanions. J Lipid Res 1970; 11:583-595), Dextran Sulphate (DS) and Manganese (II) chloride (MnCl₂) solution was used to precipitate LDL and the HDL plasma fractions. Briefly, a stock of DS and MnCl₂ were prepared as 6.5% and 2M solutions respectively. For the Total Extracellular vesicle (TEX) fraction, Xtractt buffer (Cavadis B.V.; 1:4) was added to 125 μl plasma and mixed. The mixture was incubated at 4° C. for 30 min and subsequently centrifuged at 4,800 g at 4° C. for 10 min. This pellet was dissolved in 125 μL Roche lysis buffer (Roche #04719956001) and used in the quantitative magnetic bead assays as the TEX fraction. For precipitation of the LDL fraction, DS stock (1:125) and MnCl₂ stock (1:40) were added into another 125 μL plasma and mixed. The mixed sample was centrifuged for 10 min at 4,800 g at 4° C. precipitating the LDL fraction pellet. This pellet was dissolved in 125 μL lysis buffer and used in the quantitative magnetic bead assays as the LDL fraction. For the HDL fraction, 60 μL of supernatant above the LDL pellet was transferred to a new tube topped up with 65 μL Phosphate-Buffered-Saline (PBS) and mixed. Next, DS stock (1:10) and MnCl₂ stock (1:10) were added into the 125 μl diluted supernatant and mixed. Subsequently, the sample was incubated for 2 h at 4° C. and the sample was centrifuged for 10 min at 4,800 g at 4° C. to collect the HDL fraction pellet. This pellet was dissolved in 125 μL Roche lysis buffer and used in the quantitative magnetic bead assays as the HDL fraction. For the Remaining Extracellular Vesicles (REX) fraction, 32 μl of Xtractt was added to the remaining supernatant after the HDL to precipitate the remaining extracellular vesicles. After 30 min at 4° C. followed by centrifugation at 4000 g for 30 min, the pellet was dissolved in 125 μl Roche lysis buffer with the REX vesicles lysed in this fraction.

### Proteomics

In order to determine the protein content of the vesicle sub-fractions, a mass spectrometry based proteomics run on the LDL fraction of plasma collected from MINERVA patients was performed. Liquid chromatography-tandem mass spectrometry (LC-MS/MS) was used for proteomic analysis as described (Sok Hwee Cheow et al. Simultaneous Enrichment of Plasma Soluble and Extracellular Vesicular Glycoproteins Using Prolonged Ultracentrifugation-Electrostatic Repulsion-hydrophilic Interaction Chromatography (PUC-ERLIC) Approach. MCP 2015 14(6):1657-1671). In short, LC-MS/MS was used to analyze purified proteins from the VLDL-EV fractions. After the VLDL-EV fraction was isolated from the plasma, the fraction was resolved in a lane of a 10% SDS-PAGE gel respectively and subsequently stained with Coomassie blue. The lane was excised and divided into 6 pieces equally for in-gel trypsin digestion; after reduction and alkylation, each piece was digested with Trypsin overnight. Digested peptides were extracted from the gel, dried and then desalted. The peptides were analyzed with a Q-Exactive LC-MS/MS (Thermo Scientific, San Jose, USA). The raw data were converted to mascot generic (mgf) files using Proteome Discoverer ver. 1.4 (Thermo Scientific, San Jose, USA) and then the mgf files were searched against Uniprot human proteome database using an in-house Mascot Server ver. 2.4.1 (Matrix Science, London, UK). Ingenuity Pathway Analysis (IPA®, QIAGEN Redwood City, Build Version 321501M, Content Version 17199142) was used to determine canonical pathways statistically overrepresented in the proteins compared to the Ingenuity database. Proteomics identified 815 individual proteins from which 572 could be mapped to the Ingenuity database.

The top 5 canonical pathways identified were:
LXR/RXR activation: p=8.15*10⁻⁵⁶
Acute Phase response signaling: p=4.35*10⁻⁴⁵
FXR/RXR activation: p=1.58*10⁻⁴²
Coagulation system: p=1.23*10⁻³¹
Complement system: p=5.12*10⁻²⁷

Proteins selected for verification was done on the following selection criteria: (1) Proteins described as extracellular vesicle protein in Exocarta database (http://exocarta.org/) or identified as extracellular vesicle proteins in earlier studies performed by the inventors; (2) Proteins associated with atherosclerotic disease as the underlying cause of stable coronary artery disease and unstable angina; and (3) Availability of two compatible antibody and recombinant protein that can be used in immuno-bead assay.

As a result, the inventors found the four following proteins to be of particular relevance:
CD14 (LXR/RXR activation pathway) UniProtKB—P08571 (CD14_HUMAN)
SerpinG1 (Acute Phase response pathway) UniProtKB—P05155 (IC1_HUMAN)
SerpinF2 (Acute Phase response pathway/Coagulation system) UniProtKB—P08697 (A2AP_HUMAN)
SerpinC1 (Coagulation system) UniProtKB—P01008 (ANT3_HUMAN)

Next to these four, the inventors selected Cystatin C UniProtKB—P01034 (CYTC_HUMAN), because this protein is not added to any canonical pathway but it is a vesicle protein and is associated with future cardiovascular events. Furthermore, an immuno bead assay is available that is compatible with multiplexing for SerpinG1, CD14 and SerpinF2.

### Example 2. Myomarker Stable Angina Cohort

The MYOMARKER stable coronary artery disease cohort (abbreviation for MYOcardial ischemia detection by circulating bioMARKERs) is a single center, prospective cohort study of patients, who are evaluated in the Meander Medical Center Cardiology outpatient clinic for (recent onset) suspected symptomatic coronary artery disease, undergoing radionuclide myocardial perfusion imaging (rMPI) as indicated by their own cardiologist. Ahead of rMPI, venous blood (6×10 cc) is obtained from the peripheral intravenous cannula, which is inserted as part of standard preparation for rMPI. The plasma component is frozen and stored at −80° C. within 1 hour after sample collection. All patients are evaluated and managed according to the international guidelines, by their own cardiologist. Patient enrollment started in August 2014 and continues until at least 1250 patients are participating in the study. At the filing of the present application (November 2015) over 800 patients were enrolled. Patients younger than 18 years and patients who are unable or unwilling to give their informed consent are not included in this study.

### Data Acquisition

Patient information is collected and documented in a digital case record form: clinical presentation, medical history, cardiovascular risk factors, current medication use, ECG evaluation, blood biochemical parameters and results of additional investigations.

### Endpoint Adjudication

Objectified ischemic coronary artery disease, as determined by radionuclide myocardial perfusion imaging (Rubidium-82) and adjudicated by a panel of at least two nuclear medicine physicians.

### Follow-Up

Patients are followed up for at least 1 year after inclusion. Local hospital records were checked for hospitalization (heart failure or unstable angina), additional cardiac and/or coronary imaging, coronary revascularization, myocardial infarction and death. Patients were contacted by letter and/or telephone and were asked if they had been admitted to a hospital since inclusion. When this was the case, the hospital records were checked for event verification, also when patients were admitted to a center other than the center of inclusion. Major adverse cardiovascular events (MACE) were scored, defined as non-fatal MI (STEMI/NSTEMI type 1), coronary revascularization (PCI or CABG), hospital admission for heart failure or unstable angina and/or all cause death during 1 year of follow-up.

### Preparation of Plasma Fractions and Proteomics

Preparation of plasma fractions and proteomics was performed in the same way as in example 1.

### Example 3. Localization of Proteins Associated with Extracellular Vesicles

In order to determine if the five proteins selected were present in or on extracellular vesicles (EV), density gradient ultracentrifugation was performed. VLDL-EV, HDL-EV, REX-EV and TEX-EV fractions were isolated from 8 mL plasma as mentioned under example 1. Each of the precipitates (LDL, HDL, REX, TEX) was re-suspended in 500 μl PBS and used for the density gradient centrifugation. In order to make the density gradient buffer, 5 solutions containing different concentrations of OptiPrep™ medium (OptiPrep, Axis-shiled #1114542) were prepared with 10×PBS pH 7.4 (Ambion®, Life Technologies # AM9265) and ddH₂O. The working solutions comprised 5%, 10%, 20%, 30% and 40% OptiPrep and 1×PBS respectively. The solutions were added and overlaid into the ultracentrifuge tubes (Beckman Coulter #344059) sequentially from the highest density one (with the highest concentration of OptiPrep) to the lowest density one (with the lowest concentration of OptiPrep). Subsequently, the pellets isolated of each fraction was carefully added in the ultracentrifuge tubes on top of the density gradient buffers. Then they were spun at 200,000 g for 18 h at 4° C. with low acceleration and low brake (Beckman Coulter # Optima XL-90 Ultracentrifuge # SW 41 Ti Rotor). Afterwards, 10 different gradient sub-fractions (1 mL per sub-fraction) were harvested sequentially (from top to bottom) in each ultracentrifuge tube and transferred into 1.5 mL Eppendorf tubes. After thorough vortexing of the sub-fractions, 900 μL of each sub-fraction was transferred into a new ultracentrifuge tube together with 7 mL 0.1% BSA buffer (Sigma Aldrich #05470, in 1×PBS, m/v), and then centrifuged at 200,000 g for 1 h at 4° C. The pellets were re-suspended in 200 μL Roche lysis buffer for protein level measurement.

Quantitative measurement using a beads-based immunoassay, was done as described elsewhere (Kanhai et al. Microvesicle protein levels are associated with increased risk for future vascular events and mortality in patients with clinically manifest vascular disease. Int J Cardiol 2013 168:2358-2363). In short, the beads (Luminex # MagPlex-C Microspheres, MC100) were conjugated with the selected antibodies to synthesize the bead-capture antibody complex. Samples were incubated with the bead-capture antibody complex and subsequently with the biotinylated antibodies to detect the captured protein. Streptavidin-phycoerythrin (SA-PE, BD bioscience #554061) was added to quantify the concentration of captured protein. Standard curves were correlated to the SA-PE signal and dilution of homologous recombinant proteins. The Bio-Plex® 200 Systems (Bio-Rad #171-000201) were used for measurement and data process. The antibodies and recombinant proteins used in detection were listed as follow. For SerpinC1: Antithrombin III antibody (NOVUS # NBP1-05149), human SerpinC1 Biotinylated affinity purified PAb (R&D # BAF1267), recombinant human SerpinC1 (R&D System #1267-PI-010); for SerpinF2: anti-human SerpinF2 (R&D Systems # MAB1470), biotinylated anti-human SerpinF2 (R&D Systems # BAF1470), recombinant human SerpinF2 (R&D Systems #1470-PI-010). For Cystatin C were anti-Human Cystatin C (R&D Systems # MAB11962), biotinylated anti-Human Cystatin C (R&D Systems # BAM11961) and recombinant Human Cystatin C (R&D Systems #1196-PI). For CD14, anti-Human CD14 (R&D Systems # MAB3832), biotinylated anti-Human CD14 (R&D Systems # BAF383) and recombinant Human CD14 (R&D Systems #383-CD) were used and for SerpinG1, anti-Human SerpinG1 (R&D Systems # MAB2488), biotinylated anti-Human SerpinG1 (R&D Systems # BAF2488) and recombinant Human SerpinG1 (R&D Systems #2488-PI) were used. This quantitative measurement of protein levels is also referred to as the Bioplex assay.

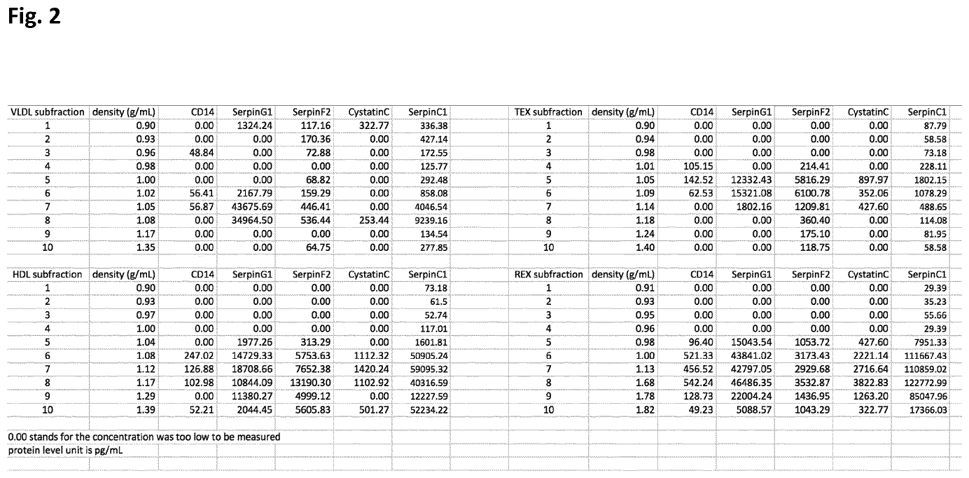

After density gradient ultracentrifugation each fraction was used in the Bioplex assay to measure the protein concentration in each fraction as well as the density in each of the fractions. For each of the 4 plasma sub-fractions and the measurements of CD14, SerpinG1, SerpinF2, Cystatin C, SerpinC1 in each of the 10 density gradient fractions is shown in FIG. 2. This table shows that the proteins are indeed associated with extracellular vesicles as only vesicles can float in such density gradients. LDL particles also float but due to the high lipid content their density is lower than 1. HDL have a density of 1-1.2. The REX and TEX fraction do not contain lipoparticles. In the table of FIG. 2 it is shown that LDL particles do not contain the EV proteins as LDL particles float all below a density <1. For HDL, this is less clear as density of EV partly coincides with HDL although the EV proteins are also present in the higher density region where no HDL particles are present. For the REX and TEX fraction, the proteins all float with extracellular vesicles with a density between 1-1.2 (TEX) and between 1-1.8.

### Example 4. Validation of Extracellular Vesicle Proteins for Diagnosis of Stable Coronary Artery Disease and Unstable Angina

### Statistical Analyses for Unstable Angina (Minerva Cohort) and Stable Angina (Myomarker Cohort)

Differences in baseline-characteristics were analyzed using Chi-square test for categorical variables, T-tests for normally distributed continuous variables and Mann-Whitney-U-tests for continuous variables that were not normally distributed. To calculate the odds ratio and enable the direct comparison between different proteins and fractions, EV-protein levels were converted into standard deviation units, or the z-score, by using the observed value minus the mean value, divided by the standard deviation. Selection of the best combination of markers and sub-fractions was done using Logistic regression analysis with forward selection based upon the Chi-square test and based upon Akaike Information Criterion (AIC). Receiver-operating characteristic (ROC) analysis was performed for AUC and optimal calculated cutoff for NPV and PPV. All statistics was achieved by SPSS® (IBM®, Version 22) and Rstudio using R software for statistical computing version 3.1.2.

### Diagnosis of Unstable Angina

From the Minerva cohort, 30 definitive unstable angina patients vs 30 matched control patients (e.g. matched for sex, age, history, diabetes, BMI, medication) were selected and SerpinF2, SerpinG1, SerpinC1, CD14, and Cystatin C were measured in all plasma fractions of LDL, HDL, REX, TEX using sequential precipitation as described under Example 1 above and an immuno-bead assay as described under example 3 above. The best performing individual marker/fraction pairs and marker/ratio pairs for identification of unstable angina are listed in table 1. Table 1 lists the most significant and best performing marker/fraction pairs and marker/ratio pairs, which on their own, and with no need for combination with further marker/fraction pairs or marker/ratio pairs, provide a statistically relevant identification of subjects suffering from unstable angina. Table 1 shows the AUC values upper and lower values obtained in the ROC plots of these most significant and best performing individual marker/fraction pairs or marker/ratio pairs. Abbreviations used in table 1 are: c1=SerpinC1, g1=SerpinG1, f2=SerpinF2, cc=Cystatin C, cd14=CD14, Id1=Low-Density lipoprotein, hdl=High-Density Lipoprotein, rex=remaining fraction, tex=total fraction. By way of example “c1 hdl/rex” means that the ROC plot is based on the ratio of a value derived from the concentration of SerpinC1 in plasma fraction HDL over plasma fraction REX, where the ROC plot is performed on the data from the above-mentioned patients and control groups of the Minerva cohort. All listed combinations have a p-value of 0.05 or less.

TABLE 1

| Markers | AUC | lower | Upper |
|---|---|---|---|
| c1-hdl/rex | 0.855 | 0.753 | 0.957 |
| cdl4-hdl/rex | 0.847 | 0.746 | 0.948 |
| c1-hdl | 0.844 | 0.741 | 0.947 |
| cdl4-hdl/tex | 0.838 | 0.727 | 0.948 |
| c1-ldl/hdl | 0.827 | 0.717 | 0.937 |
| c1-hdl/tex | 0.826 | 0.705 | 0.947 |
| cdl4-ldl/hdl | 0.825 | 0.71l | 0.939 |
| cdl4-hdl | 0.788 | 0.661 | 0.915 |
| cdl4-tex | 0.708 | 0.571 | 0.845 |
| cc-hdl/rex | 0.691 | 0.55 | 0.831 |
| cdl4-rex | 0.675 | 0.535 | 0.815 |
| cc-hdl/tex | 0.673 | 0.532 | 0.813 |
| f2-rex/tex | 0.665 | 0.523 | 0.808 |
| cdl4-ldl/tex | 0.663 | 0.518 | 0.807 |
| cc-ldl/hdl | 0.655 | 0.507 | 0.802 |
| f2-tex | 0.646 | 0.502 | 0.791 |

As apparent from table 1, the three best individual markers are SerpinC1 with the ratio HDL/REX and an AUC of 0.855 (95% Cl 0.753-0.957); CD14 with the ratio HDL/REX and an AUC of 0.847 (95% Cl 0.746-0.948) and SerpinC1 in the HDL fraction with an AUC of 0.844 (95% Cl 0.741-0.947) to discriminate between unstable angina and matched controls.

The best performing combinations of two marker/fraction pairs and marker/ratio pairs for identification of unstable angina are listed in table 2. Combination of two marker/fractions and/or marker/ratios means that two different values derived from the concentration of at least one protein marker is determined. The two different values can for example be values derived from the concentration of the same marker but in different fractions and/or ratios or it can be of different markers in the same or different fractions and/or ratios. Table 2 lists the most significant and best performing combinations of two marker/fraction pairs and marker/ratio pairs and show the AUC values as obtained in the ROC plots of these most significant and best performing combinations of marker/fraction pairs and/or marker/ratio pairs. ROC plots are performed on the data from the above-mentioned patient and control groups of the Minerva cohort. All listed combinations have a p-value of 0.05 or less. Abbreviations used in table 2 are the same as for table 1. However, in table 2 the “/” sign is missing between the fractions of which a ratio is taken. By way of example, “c1ldlhdl” means the ratio of a value derived from the concentration of SerpinC1 in fraction LDL over HDL.

| Markers | AUC |
|---|---|
| c1ldlhdl + c1hdltex | 0.902 |
| cdl4hdlrex + c1ldlrex | 0.9 |
| cdl4tex + c1hdl | 0.896 |
| cdl4hdlrex + c1ldlhdl | 0.895 |
| cdl4hdltex + c1ldlhdl | 0.892 |
| cdl4hdlrex + c1ldl | 0.891 |
| c1hdlrex + ccrextex | 0.89 |
| c1ldlrex + c1hdlrex | 0.888 |
| c1hdlrex + ccldltex | 0.888 |
| c1ldlhdl + c1hdlrex | 0.886 |
| c1tex + c1hdltex | 0.885 |
| c1ldltex + c1hdltex | 0.885 |
| cdl4hdlrex + g1ldlhdl | 0.884 |
| c1ldlhdl + cchdltex | 0.884 |
| c1ldl + c1hdlrex | 0.883 |
| cdl4hdltex + c1hdlrex | 0.882 |
| cdl4tex + c1hdlrex | 0.881 |
| cdl4hdltex + g1ldlhdl | 0.881 |
| c1hdl + ccldlhdl | 0.881 |
| c1hdlrex + g1ldltex | 0.881 |
| c1hdlrex + g1hdltex | 0.881 |
| c1hdlrex + g1rextex | 0.881 |
| cdl4hdltex + c1tex | 0.879 |
| c1ldlhdl + c1ldltex | 0.879 |
| c1hdlrex + c1hdltex | 0.879 |
| c1hdltex + c1rextex | 0.878 |
| cdl4hdlrex + cdl4hdltex | 0.877 |
| c1hdlrex + c1rextex | 0.877 |
| c1hdlrex + f2rextex | 0.877 |
| c1hdlrex + ccldlhdl | 0.877 |
| cdl4tex + cdl4hdlrex | 0.876 |
| c1hdlrex + g1ldlrex | 0.876 |
| c1hdlrex + cctex | 0.876 |
| cdl4hdlrex + glhdltex | 0.875 |
| cdl4hdltex + cdl4rextex | 0.875 |
| c1ldl + c1hdl | 0.875 |
| cdl4hdlrex + c1hdltex | 0.873 |
| cdl4hdlrex + g1ldlrex | 0.873 |
| c1hdl + c1ldlhdl | 0.873 |
| c1hdl + g1hdltex | 0.873 |
| c1ldlhdl + g1ldltex | 0.873 |
| cd14ldlhdl + c1ldlhdl | 0.872 |
| cdl4hdlrex + ccrextex | 0.872 |
| c1ldl + c1ldlhdl | 0.872 |
| c1hdl + ccldltex | 0.872 |
| cdl4hdlrex + cdl4rextex | 0.871 |
| c1hdl + g1ldltex | 0.871 |
| c1hdlrex + cchdltex | 0.871 |
| cdl4hdl + c1ldlhdl | 0.87 |
| cd14ldlrex + c1ldlhdl | 0.87 |
| cdl4hdlrex + ccldlhdl | 0.87 |
| cdl4hdltex + c1hdl | 0.87 |
| cdl4hdltex + c1ldltex | 0.87 |
| c1ldlhdl + c1ldlrex | 0.87 |
| c1hdlrex + f2hdltex | 0.87 |
| c1hdlrex + g1ldlhdl | 0.87 |
| c1hdlrex + g1ldl | 0.869 |
| c1hdl + glrextex | 0.867 |
| c1hdl + ccrextex | 0.867 |
| c1ldlhdl + f2hdl | 0.867 |
| cd14ldlhdl + c1hdlrex | 0.866 |
| cdl4hdltex + g1ldlrex | 0.866 |
| cdl4rextex + c1hdlrex | 0.866 |
| c1tex + c1hdlrex | 0.866 |
| c1ldltex + c1hdlrex | 0.866 |
| c1hdlrex + f2tex | 0.866 |
| cdl4rex + c1ldlhdl | 0.865 |
| cdl4tex + c1ldlhdl | 0.865 |
| cdl4hdlrex + ccldltex | 0.865 |
| cdl4hdltex + c1lcll | 0.865 |
| c1hdl + c1rextex | 0.865 |
| c1hdl + f2tex | 0.865 |
| cdl4hdlrex + f2rextex | 0.864 |
| cdl4hdltex + c1ldlrex | 0.864 |
| c1ldlhdl + ccldltex | 0.864 |
| c1hdl + g1ldl | 0.863 |
| c1hdl + g1ldlhdl | 0.863 |
| c1hdl + g1ldlrex | 0.863 |
| c1hdlrex + f2hdl | 0.863 |
| c1hdlrex + g1hdlrex | 0.863 |
| c1hdlrex + cchdlrex | 0.863 |
| cdl4hdlrex + c1rextex | 0.861 |
| cdl4hdlrex + glrextex | 0.861 |
| c1hdl + c1hdltex | 0.861 |
| c1hdl + cctex | 0.861 |
| c1hdlrex + f21dlrex | 0.861 |
| c1hdlrex + f2idltex | 0.861 |
| c1hdlrex + ccrex | 0.861 |
| cdl4hdlrex + c1hdl | 0.86 |
| cdl4hdlrex + c1hdlrex | 0.86 |
| c1hdl + c1rex | 0.86 |
| c1ldlrex + c1hdltex | 0.86 |
| c1hdlrex + ccldl | 0.86 |
| cdl4hdl + cdl4tex | 0.859 |
| cdl4hdl + c1hdlrex | 0.859 |
| cd14ldlhdl + ccldltex | 0.859 |
| cdl4hdlrex + f2hdl | 0.859 |
| cdl4hdlrex + cchdlrex | 0.859 |
| c1ldlhdl + g1hdltex | 0.859 |
| c1ldlhdl + ccrextex | 0.859 |
| cdl4hdlrex + f2tex | 0.858 |
| c1hdl + c1hdlrex | 0.858 |
| c1hdl + f2hdltex | 0.858 |
| c1rex + c1hdlrex | 0.858 |
| c1ldlhdl + g1rextex | 0.858 |
| cdl4tex + cd14ldlhdl | 0.857 |
| cdl4hdlrex + g1lc1l | 0.857 |
| cdl4hdlrex + ccrex | 0.857 |
| c1hdl + f2hdl | 0.857 |
| cd14ldlhdl + cdl4hdlrex | 0.856 |
| cd14ldlrex + cdl4hdlrex | 0.856 |
| cd14ldlrex + c1hdlrex | 0.856 |
| cd14ldltex + c1ldlhdl | 0.856 |
| cd14ldlhdl + cd14ldlrex | 0.855 |
| cd14ldltex + c1hdlrex | 0.855 |
| cdl4hdltex + c1rextex | 0.855 |
| c1ldl + c1hdltex | 0.855 |
| c1hdl + f2rextex | 0.855 |
| c1hdl + cchdltex | 0.855 |
| c1hdlrex + g1hdl | 0.855 |
| c1hdlrex + g1tex | 0.855 |
| cdl4hdl + cdl4rex | 0.854 |
| cdl4rex + cdl4hdlrex | 0.854 |
| cdl4rex + c1hdlrex | 0.854 |
| cd14ldlhdl + c1hdl | 0.854 |
| cd14ldlhdl + c1hdltex | 0.854 |
| cdl4hdlrex + f2hdlrex | 0.854 |
| cdl4hdlrex + glldltex | 0.854 |
| cdl4hdlrex + ccldl | 0.854 |
| c1hdlrex + cchdl | 0.854 |
| c1hdlrex + ccldlrex | 0.854 |
| c1hdltex + ccldlrex | 0.854 |
| cdl4hdlrex + cctex | 0.853 |
| cdl4hdlrex + cchdltex | 0.853 |
| cdl4hdltex + g1lc1l | 0.853 |
| c1hdl + c1ldlrex | 0.853 |
| c1hdl + c1ldltex | 0.853 |
| c1hdl + g1hdlrex | 0.853 |
| c1hdlrex + f2rex | 0.853 |
| c1hdlrex + f2hdlrex | 0.853 |
| c1hdltex + g1ldlhdl | 0.853 |
| cd14ldl + c1hdl | 0.852 |
| cdl4rex + cd14ldlhdl | 0.852 |
| cd14ldltex + c1hdl | 0.852 |
| cdl4hdlrex + c1rex | 0.852 |
| cdl4hdlrex + c1ldltex | 0.852 |
| cdl4hdlrex + f2ldltex | 0.852 |
| c1hdl + ccldlrex | 0.852 |
| c1ldlhdl + f2rextex | 0.852 |
| cd14ldl + cdl4hdlrex | 0.851 |
| cd14ldl + c1hdlrex | 0.851 |
| cd14hdl + cdl4hdlrex | 0.851 |
| cd141dlhdl + cchdltex | 0.851 |
| cdl4hdlrex + f2ldlrex | 0.851 |
| cdl4hdlrex + gltex | 0.851 |
| cdl4hdltex + glhdltex | 0.851 |
| c1hdl + g1tex | 0.851 |
| c1hdlrex + f21dl | 0.851 |
| c1hdlrex + g1rex | 0.851 |
| cd14ldlhdl + g1hdltex | 0.85 |
| cd14ldlhdl + cctex | 0.85 |
| cd14hdlrex + f2hdltex | 0.849 |
| cd14hdlrex + g1hdlrex | 0.849 |
| c1ldlhdl + f2ldltex | 0.849 |
| c1ldlhdl + ccldlhdl | 0.849 |
| c1ldlhdl + cchdlrex | 0.849 |
| cdl4rex + c1hdltex | 0.848 |
| cdl4tex + cdl4hdltex | 0.848 |
| cdl4hdlrex + g1hdl | 0.848 |
| cdl4hdlrex + g1rex | 0.848 |
| c1ldlhdl + cctex | 0.848 |
| cd14ldltex + cdl4hdltex | 0.847 |
| cdl4hdlrex + c1tex | 0.847 |
| cdl4hdlrex + f2rex | 0.847 |
| cdl4hdlrex + cchdl | 0.847 |
| cdl4rextex + c1hdl | 0.847 |
| c1hdl + f2rex | 0.847 |
| c1hdl + g1rex | 0.847 |
| c1ldlhdl + f2tex | 0.847 |
| c1hdltex + g1ldlrex | 0.847 |
| cdl4hdlrex + f21dlhdl | 0.846 |
| cdl4hdlrex + ccldlrex | 0.846 |
| c1hdl + f2ldltex | 0.846 |
| c1hdl + g1hdl | 0.846 |
| c1hdl + ccldl | 0.846 |
| cd14ldlhdl + cdl4hdltex | 0.845 |
| cd14ldlhdl + ccrextex | 0.845 |
| cd14hdltex + glhdlrex | 0.845 |
| c1hdl + c1tex | 0.845 |
| c1hdl + f2hdlrex | 0.845 |
| c1hdl + ccrex | 0.845 |
| c1ldlhdl + f2rex | 0.845 |
| cd14ldlhdl + ccldlhdl | 0.844 |
| cd14ldlrex + c1hdl | 0.844 |
| c1ldlhdl + f2hdltex | 0.843 |
| c1hdltex + g1hdlrex | 0.843 |
| cd14ldlhdl + c1lcll | 0.842 |
| cd14ldlhdl + g1ldlrex | 0.842 |
| cd14ldlhdl + g1rextex | 0.842 |
| cd14ldltex + cd14hdlrex | 0.842 |
| cd14hdltex + g1rextex | 0.842 |
| c1hdl + cchdl | 0.842 |
| c1hdlrex + f2ldlhdl | 0.842 |
| cd14ldlhdl + ccldlrex | 0.841 |
| cd14hdlrex + f2ldl | 0.841 |
| cd14hdltex + f2tex | 0.841 |
| cd14hdltex + g1hdl | 0.841 |
| cd14hdltex + ccldl | 0.841 |
| cd14hdltex + ccldlrex | 0.841 |
| c1hdltex + ccldlhdl | 0.841 |
| cd14hdl + c1hdl | 0.84 |
| cd14rex + c1hdl | 0.84 |
| cd141dlhdl + g1ldlhdl | 0.84 |
| cd14hdltex + f2hdlrex | 0.84 |
| cd14hdltex + cctex | 0.84 |
| cd14hdltex + ccldltex | 0.84 |
| c1hdl + f2ldl | 0.84 |
| c1hdl + cchdlrex | 0.84 |
| c1ldlhdl + g1hdlrex | 0.84 |
| cd14ldlhdl + cd14ldltex | 0.839 |
| cd14hdltex + f2hdltex | 0.839 |
| cd14hdltex + glldltex | 0.839 |
| c1hdl + f2ldlhdl | 0.839 |
| c1hdltex + cchdlrex | 0.839 |
| cd14hdltex + g1rex | 0.838 |
| cd14hdltex + g1tex | 0.838 |
| cd14rextex + c1hdltex | 0.838 |
| c1rex + c1ldlhdl | 0.838 |
| c1hdltex + f2hdl | 0.838 |
| cd14ldl + cdl4hdltex | 0.837 |
| cd14ldlhdl + g1tex | 0.836 |
| cd14ldlrex + cdl4hdltex | 0.836 |
| cd14ldl rex + c1hdltex | 0.836 |
| cd14hdltex + f2rextex | 0.836 |
| cd14hdltex + ccrex | 0.836 |
| cd141dl + c1hdltex | 0.835 |
| cd14hdl + c1lc1l | 0.835 |
| cd14ldlhdl + c1ldlrex | 0.835 |
| cd14ldlhdl + f21dlhdl | 0.835 |
| cd14hdltex + cchdlrex | 0.835 |
| c1hdl + f2ldlrex | 0.835 |
| c1ldlhdl + ccldlrex | 0.835 |
| cd14ldlhdl + g1ldltex | 0.834 |
| cd14rextex + c1ldlhdl | 0.834 |
| c1ldlhdl + ccldl | 0.834 |
| cd14hdl + c1hdltex | 0.833 |
| cd14rex + cdl4hdltex | 0.833 |
| cd14tex + c1hdltex | 0.833 |
| cd14hdltex + c1rex | 0.833 |
| cd14hdltex + f2rex | 0.833 |
| cd14hdltex + f2ldltex | 0.833 |
| c1hdltex + f2rex | 0.833 |
| cdl4hdltex + f2hdl | 0.832 |
| c1rex + c1hdltex | 0.832 |
| cd14ldlhdl + f2ldlrex | 0.831 |
| cd14hdltex + c1hdltex | 0.83 |
| cd14hdltex + cchdltex | 0.83 |
| cd14hdltex + ccrextex | 0.83 |
| c1ldlhdl + g1tex | 0.83 |
| c1ldlhdl + g1ldlrex | 0.83 |
| c1hdltex + g1lcll | 0.83 |
| cd14ldlhdl + g1hdlrex | 0.829 |
| c1ldlhdl + f2ldlhdl | 0.829 |
| c1hdltex + f2tex | 0.829 |
| c1hdltex + g1tex | 0.829 |
| c1hdltex + cchdl | 0.829 |
| cd14ldl + cd14ldlhdl | 0.828 |
| cd14ldlhdl + cd14rextex | 0.828 |
| cd14ldlhdl + c1tex | 0.828 |
| cd14ldlhdl + c1ldltex | 0.828 |
| cd14ldlhdl + c1rextex | 0.828 |
| cd14ldlhdl + cchdl | 0.828 |
| cd14ldltex + c1hdltex | 0.828 |
| cd14hdltex + f2ldlhdl | 0.828 |
| cd14hdltex + f2ldlrex | 0.828 |
| cd14hdltex + ccldlhdl | 0.828 |
| c1ldlhdl + c1rextex | 0.828 |
| c1ldlhdl + f2ldl | 0.828 |
| c1ldlhdl + cchdl | 0.828 |
| c1hdltex + f2ldlhdl | 0.828 |
| c1hdltex + g1ldltex | 0.828 |
| c1hdltex + ccldltex | 0.828 |
| c1hdltex + cchdltex | 0.828 |
| c1ldlhdl + f2hdlrex | 0.827 |
| c1ldlhdl + g1rex | 0.827 |
| c1hdltex + f2hdlrex | 0.827 |
| c1hdltex + f2hdltex | 0.827 |
| c1hdltex + f2rextex | 0.827 |
| c1hdltex + g1rex | 0.827 |
| cd14ldl + cdl4hdl | 0.826 |
| cd14ldl + c1ldlhdl | 0.826 |
| cd14hdl + cd14ldlhdl | 0.826 |
| cd14ldlhdl + g1hdl | 0.826 |
| cd14ldlhdl + cchdlrex | 0.826 |
| cd14hdltex + cchdl | 0.826 |
| c1ldlhdl + f2ldlrex | 0.826 |
| c1ldlhdl + g1hdl | 0.826 |
| c1ldlhdl + g1ldlhdl | 0.826 |
| c1hdltex + g1rextex | 0.826 |
| c1hdltex + ccrex | 0.826 |
| c1hdltex + cctex | 0.826 |
| c1hdltex + ccrextex | 0.826 |
| cd14ldlhdl + f2tex | 0.825 |
| cd14ldlhdl + g1lcll | 0.825 |
| cd14ldlhdl + g1rex | 0.825 |
| cd14ldlhdl + ccldl | 0.825 |
| cd14ldlhdl + ccrex | 0.825 |
| cd14hdltex + f2ldl | 0.825 |
| cd14hdl + cctex | 0.824 |
| cd14ldlhdl + f2ldltex | 0.824 |
| cd14ldlhdl + f2hdltex | 0.824 |
| cd14ldl hdl + f2rextex | 0.824 |
| c1ldlhdl + g1lcll | 0.824 |
| c1ldlhdl + ccrex | 0.824 |
| c1hdltex + ccldl | 0.824 |
| cd14hdl + g1ldlhdl | 0.823 |
| cd14ldlhdl + f2hdlrex | 0.823 |
| c1hdltex + f2ldlrex | 0.823 |
| c1tex + c1ldlhdl | 0.822 |
| c1hdltex + g1hdltex | 0.822 |
| c1hdltex + f2ldl | 0.821 |
| cd14hdl + ccldlrex | 0.82 |
| cd14ldlhdl + f2hdl | 0.82 |
| cd14ldlhdl + f2rex | 0.82 |
| c1hdltex + g1hdl | 0.82 |
| cd14ldlhdl + c1rex | 0.819 |
| c1hdltex + f2ldltex | 0.819 |
| cd14hdl + cdl4hdltex | 0.818 |
| cd14ldlhdl + f2ldl | 0.818 |
| cd14hdl + c1ldlrex | 0.816 |
| cd14hdl + ccldltex | 0.814 |
| cd14hdl + g1ldlrex | 0.808 |
| cd14hdl + cchdl | 0.808 |
| cd14hdl + f2tex | 0.806 |
| cd14hdl + ccldlhdl | 0.805 |
| cd14hdl + g1hdltex | 0.804 |
| cd14hdl + glrextex | 0.803 |
| cd14hdl + f2hdl | 0.802 |
| cd14hdl + f2rex | 0.8 |
| cd14hdl + cchdlrex | 0.8 |

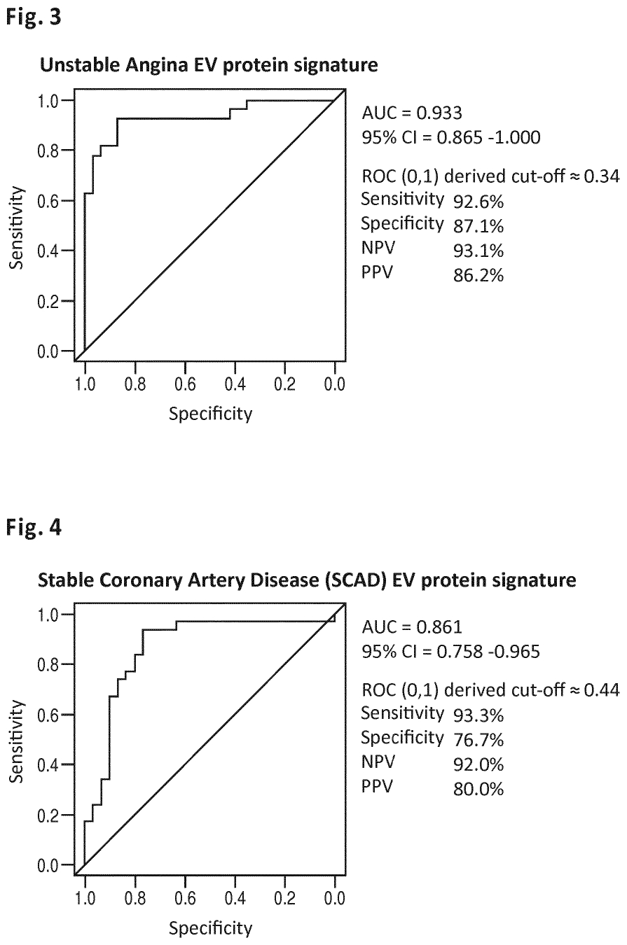

Selection of the best combination of markers and sub-fractions without using the ratios of markers between the sub-fractions using Logistic regression analysis with forward selection based upon the Chi-square test showed an AUC of 0.933. The combination of markers consists of SerpinC1-HDL+CD14-TEX+SerpinC1-LDL (FIG. 3), without using the ratios between the sub-fractions, because with 30 vs 30 patients one is underpowered to use all biomarkers in all combinations. This is a conservative choice that prevents overfitting of the regression model and for this gives a more realistic calculation. With an optimal cut-off value based on the AUC, the sensitivity of these 3 markers was 92.6% and specificity of 87%. The Negative Predictive Value (NPV) is 93.1% and the Positive Predictive Value is 86.2%. This means that measuring these three extracellular protein levels in the LDL (SerpinC1), HDL (SerpinC1) and TEX (CD14) plasma sub-fraction can diagnose unstable angina accurately based on a blood sample of less than 250 μl. With an NPV of 93.1% it can also determine which patients can be send home safely. Since the UA patients are matched with the controls, the influence of potential confounders can be excluded. The highest scoring combinations of marker/fraction using the Minerva cohort as discussed here, are provided in FIG. 6 but now including the ratios of the biomarker between the plasma sub-fractions. This combination/ratios between fractions will be used in the calculation of the best combination of extracellular vesicle markers after validation of the extracellular vesicle markers in the large cohorts (Example 5) that will give enough power as patient numbers are much higher.

### Diagnosis of Stable Angina

Although unstable angina is thought to be much more coagulation-associated then stable angina, the underlying disease process is the same: atherosclerosis.

For this, the 5 extracellular vesicle proteins SerpinF2, SerpinG1, SerpinC1, CD14, and Cystatin C were measured in the plasma fractions LDL, HDL, REX and TEX in a cohort of 30 stable coronary artery disease patients of the Myomarker cohort and 30 sex-, age, history-, and medication-matched controls of the same cohort using sequential precipitation as described under example 1 above and an immuno-bead assay as described under example 3 above. The best performing individual marker/fraction pairs and marker/ratio pairs for identification of stable angina are listed in table 3. Table 3 lists the most significant and best performing marker/fraction pairs and marker/ratio pairs, which on their own, and with no need for combination with further marker/fraction pairs or marker/ratio pairs, provide a statistically relevant identification of subjects suffering from stable angina. Table 3 shows the AUC values upper and lower values obtained in the ROC plots of these most significant and best performing individual marker/fraction pairs or marker/ratio pairs. The ROC plots are performed on the data from the above-mentioned patient and control groups of the Myomarker cohort. All listed combinations have a p-value of 0.05 or less. The abbreviations and the nomenclature of table 3 is the same as in table 1.

TABLE 3

| Markers | AUC | Lower | upper |
|---|---|---|---|
| c1-hdl/tex | 0.842 | 0.741 | 0.944 |
| c1-hdl/rex | 0.828 | 0.718 | 10.938 |
| c1-hdl | 0.812 | 0.7 | 0.924 |
| f2-ldl/hdl | 0.81l | 0.695 | 0.927 |
| cd14-hdl/tex | 0.806 | 0.692 | 0.919 |
| cd14-1dl/hdl | 0.764 | 0.638 | 0.891 |
| cd14-hdl/rex | 0.733 | 0.605 | 0.862 |
| c1-rex/tex | 0.727 | 0.598 | 0.855 |
| c1-ldl/hdl | 0.719 | 0.589 | 0.849 |
| f2-hdl/tex | 0.717 | 0.584 | 0.851 |
| cd14-hdl | 0.716 | 0.584 | 0.847 |
| c1-tex | 0.702 | 0.567 | 0.837 |
| f2-ldl/rex | 0.689 | 0.546 | 0.832 |
| cc-hdl/tex | 0.683 | 0.547 | 0.819 |
| g1-rex/tex | 0.662 | 0.52 | 0.804 |
| cc-hdl/rex | 0.654 | 0.509 | 0.8 |
| cc-ldl/hdl | 0.653 | 0.513 | 0.794 |

The best performing combinations of two marker/fraction pairs and marker/ratio pairs for identification of stable angina are listed in table 4. Combination of two marker/fractions and/or marker/ratios means that two different values derived from the concentration of at least one protein marker is determined. The two different values can for example be values derived from the concentration of the same marker but in different fractions and/or ratios or it can be of different markers in the same or different fractions and/or ratios. Table 4 lists the most significant and best performing combinations of two marker/fraction pairs and marker/ratio pairs and show the AUC values as obtained in the ROC plots of these most significant and best performing combinations of marker/fraction pairs and/or marker/ratio pairs. The ROC plots were performed on the data from the above-mentioned patient and control groups of the Myomarker cohort. All listed combinations have a p-value of 0.05 or less. The abbreviations and the nomenclature of table 4 is the same as in table 2.

TABLE 4

| Markers | AUC |
|---|---|
| c1hdl + f21dlhdl | 0.881 |
| c1hdltex + cchdltex | 0.876 |
| cd14ldltex + c1hdltex | 0.874 |
| cd14rextex + c1hdltex | 0.873 |
| c1hdltex + ccldltex | 0.873 |
| c1hdltex + c1rextex | 0.873 |
| c1hdlrex + f2ldlhdl | 0.872 |
| f2ldlhdl + g1hdl | 0.872 |
| c1hdltex + g1rextex | 0.869 |
| f2ldlhdl + g1ldl | 0.868 |
| c1tex + c1hdlrex | 0.867 |
| 12ldl + f2hdl | 0.861 |
| c1hdlrex + c1hdltex | 0.86 |
| c1hdltex + ccrextex | 0.859 |
| c1hdl + c1tex | 0.859 |
| c1hdltex + g1hdltex | 0.857 |
| f2rex + f2ldlhdl | 0.857 |
| c1hdlrex + g1rextex | 0.857 |
| c1hdlrex + c1rextex | 0.856 |
| c1ldlhdl + f2ldlhdl | 0.856 |
| f2hdl + f21dlhdl | 0.854 |
| c1hdltex + f2ldltex | 0.853 |
| f2ldlhdl + g1ldlrex | 0.853 |
| c1hdlrex + g1hdl | 0.852 |
| cd14hdltex + c1hdlrex | 0.852 |
| c1hdlrex + f2rextex | 0.852 |
| f2ldlhdl + g1hdlrex | 0.852 |
| cd14hdlrex + c1hdl | 0.851 |
| f2ldl + f2ldlhdl | 0.851 |
| c1rex + c1hdlrex | 0.85 |
| c1hdlrex + g1hdltex | 0.85 |
| c1hdlrex + g1ldltex | 0.85 |
| cd14ldlhdl + c1hdlrex | 0.85 |
| cd14hdltex + c1hdltex | 0.849 |
| c1ldlrex + c1hdlrex | 0.849 |
| c1hdlrex + ccldltex | 0.849 |
| c1hdlrex + cchdltex | 0.849 |
| cd14ldltex + cd14hdltex | 0.848 |
| f2ldlhdl + g1rex | 0.848 |
| c1hdl + c1hdltex | 0.847 |
| c1hdltex + f2hdl | 0.847 |
| cd14rextex + c1hdlrex | 0.847 |
| c1rex + f2ldlhdl | 0.847 |
| c1hdlrex + g1tex | 0.847 |
| c1hdltex + g1ldltex | 0.846 |
| c1hdltex + f2ldlhdl | 0.845 |
| c1ldlhdl + c1hdlrex | 0.844 |
| f2ldltex + f2hdltex | 0.844 |
| cd14hdl + f2ldlhdl | 0.844 |
| cd14ldlhdl + c1hdltex | 0.844 |
| c1hdlrex + ccrex | 0.844 |
| c1hdlrex + ccrextex | 0.844 |
| c1ldl + c1hdltex | 0.843 |
| cd14hdl + c1hdlrex | 0.843 |
| c1hdltex + g1hdlrex | 0.843 |
| cdl4hdlrex + c1hdlrex | 0.843 |
| c1hdlrex + cctex | 0.843 |
| cd14ldltex + c1hdlrex | 0.843 |
| cd14ldlrex + c1hdlrex | 0.843 |
| c1hdltex + f2tex | 0.842 |
| c1hdltex + f2hdltex | 0.841 |
| c1hdltex + f2rextex | 0.841 |
| c1tex + c1hdltex | 0.841 |
| c1hdlrex + g1rex | 0.841 |
| c1hdltex + f2rex | 0.84 |
| c1hdl + c1hdlrex | 0.84 |
| cd14hdlrex + f2ldlhdl | 0.839 |
| c1ldlhdl + c1ldlrex | 0.839 |
| c1hdltex + ccldlrex | 0.838 |
| c1hdlrex + ccldlhdl | 0.838 |
| c1hdlrex + f2tex | 0.838 |
| c1hdlrex + g1hdlrex | 0.838 |
| c1ldltex + c1hdltex | 0.837 |
| cdl4rex + c1hdlrex | 0.837 |
| c1hdltex + ccldl | 0.836 |
| c1hdl + g1tex | 0.836 |
| c1hdlrex + g1ldlhdl | 0.836 |
| c1hdlrex + g1ldl | 0.836 |
| cd14ldl + c1hdltex | 0.834 |
| c1hdltex + ccrex | 0.834 |
| c1ldl + c1hdlrex | 0.834 |
| c1hdltex + g1ldlrex | 0.834 |
| c1hdlrex + f2hdl | 0.834 |
| c1hdlrex + g1ldlrex | 0.834 |
| c1hdlrex + ccldlrex | 0.834 |
| cd14tex + c1hdlrex | 0.834 |
| f2ldlhdl + cchdlrex | 0.833 |
| c1hdlrex + f2hdlrex | 0.832 |
| c1ldltex + c1hdlrex | 0.832 |
| c1hdlrex + cchdl | 0.832 |
| cdl4hdltex + c1hdl | 0.831 |
| c1hdlrex + f2rex | 0.831 |
| c1hdlrex + ccldl | 0.831 |
| c1hdlrex + cchdlrex | 0.831 |
| cd14ldl + f2ldlhdl | 0.831 |
| c1hdltex + g1hdl | 0.83 |
| c1rex + c1hdltex | 0.83 |
| cd14ldl + c1hdlrex | 0.83 |
| f2ldlhdl + g1rextex | 0.83 |
| c1hdl + f2rextex | 0.829 |
| c1hdl + c1rextex | 0.829 |
| c1hdl + cchdltex | 0.829 |
| c1hdl + g1ldltex | 0.829 |
| c1hdlrex + f2ldltex | 0.829 |
| cd14ldlhdl + c1hdl | 0.828 |
| c1hdl + c1rex | 0.828 |
| c1hdltex + g1ldlhdl | 0.827 |
| c1hdl + g1hdltex | 0.827 |
| c1tex + f2ldlhdl | 0.827 |
| c1ldlrex + c1hdltex | 0.826 |
| cd14rex + c1hdltex | 0.826 |
| cd14rextex + c1hdl | 0.826 |
| c1hdl + g1ldlhdl | 0.826 |
| cd141dl + c1hdl | 0.826 |
| cd14tex + c1hdl | 0.826 |
| cd141dlhdl + f21dlhdl | 0.825 |
| c1hdltex + g1ldl | 0.824 |
| c1hdltex + g1rex | 0.824 |
| c1hdl + g1rextex | 0.824 |
| f2ldlhdl + ccldlrex | 0.824 |
| cd14ldlrex + f2ldlhdl | 0.824 |
| f2tex + f2ldlhdl | 0.824 |
| c1hdltex + f2hdlrex | 0.823 |
| cdl4hdlrex + c1hdltex | 0.823 |
| c1hdl + ccldltex | 0.823 |
| c1hdl + ccrextex | 0.823 |
| cd14rex + c1hdl | 0.823 |
| cd141dltex + c1hdl | 0.823 |
| c1hdl + c1ldltex | 0.823 |
| cd14hdltex + f2ldlhdl | 0.823 |
| f2ldlhdl + f2hdltex | 0.823 |
| c1hdlrex + f2ldlrex | 0.823 |
| c1ldlrex + f2ldlhdl | 0.823 |
| f2ldlhdl + g1tex | 0.823 |
| f2ldlhdl + cctex | 0.823 |
| cd14ldlhdl + c1ldlhdl | 0.822 |
| c1hdlrex + f2hdltex | 0.822 |
| c1hdltex + g1tex | 0.821 |
| f2ldlhdl + f2ldltex | 0.821 |
| f2ldlhdl + g1ldltex | 0.821 |
| c1hdltex + ccldlhdl | 0.821 |
| cd14ldlrex + c1hdltex | 0.821 |
| c1hdlrex + f2ldl | 0.821 |
| cd14hdl + cdl4tex | 0.82 |
| cd141dlrex + c1hdl | 0.82 |
| c1rextex + f2ldlhdl | 0.82 |
| f2ldlhdl + cchdl | 0.82 |
| f2ldlhdl + f2rextex | 0.82 |
| cd14hdltex + ccrextex | 0.819 |
| f2ldlhdl + g1hdltex | 0.819 |
| cd14tex + f2ldlhdl | 0.817 |
| c1hdl + cctex | 0.817 |
| f2ldlrex + f2hdlrex | 0.817 |
| c1hdl + cchdlrex | 0.817 |
| c1hdl + g1ldl | 0.816 |
| c1hdl + g1hdlrex | 0.816 |
| c1hdl + cchdl | 0.816 |
| f2ldlhdl + ccldltex | 0.816 |
| f2ldlhdl + ccldlhdl | 0.816 |
| f2ldlhdl + ccrex | 0.815 |
| c1hdl + g1rex | 0.815 |
| f2ldlhdl + g1ldlhdl | 0.815 |
| c1hdl + ccrex | 0.814 |
| c1hdl + g1hdl | 0.814 |
| c1hdl + g1ldlrex | 0.813 |
| c1hdl + ccldl | 0.813 |
| f2ldlhdl + cchdltex | 0.813 |
| c1hdl + f2hdlrex | 0.813 |
| f2ldlhdl + ccldl | 0.813 |
| c1hdl + f2ldltex | 0.813 |
| f2ldlhdl + f2ldlrex | 0.813 |
| f2ldlhdl + f2hdlrex | 0.813 |
| cd14tex + c1hdltex | 0.812 |
| c1hdl + f2rex | 0.812 |
| c1hdltex + cctex | 0.812 |
| cd14ldlhdl + c1rextex | 0.812 |
| cd14ldltex + f2ldlhdl | 0.812 |
| f2ldlhdl + ccrextex | 0.812 |
| c1ldltex + f2ldlhdl | 0.812 |
| c1ldl + c1hdl | 0.81l |
| c1hdl + f2hdl | 0.81l |
| c1hdl + ccldlrex | 0.81l |
| cd14hdltex + g1rextex | 0.81l |
| c1hdl + f2tex | 0.81l |
| cd14rextex + f2ldlhdl | 0.81l |
| cd14rex + f2ldlhdl | 0.81l |
| c1ldl + f2ldlhdl | 0.81l |
| cd14hdltex + ccldltex | 0.81l |
| cd14hdl + c1hdltex | 0.81 |
| cd14hdl + c1hdl | 0.81 |
| c1hdl + ccldlhdl | 0.81 |
| cd14hdltex + g1ldltex | 0.809 |
| c1hdltex + cchdl | 0.808 |
| cd14ldlhdl + g1hdltex | 0.808 |
| cd14ldl + cd14hdltex | 0.806 |
| cd14ldlhdl + cd14hdltex | 0.806 |
| cd14ldlhdl + g1ldltex | 0.806 |
| c1hdl + c1ldlhdl | 0.804 |
| cdl4hdltex + g1hdltex | 0.803 |
| cd14ldlhdl + c1tex | 0.803 |
| cd14ldlhdl + g1rextex | 0.803 |
| c1ldl + c1ldlhdl | 0.801 |
| c1hdl + f2hdltex | 0.801 |
| cd14ldlhdl + cd14ldltex | 0.801 |
| cd14hdltex + g1ldlhdl | 0.8 |
| c1hdl + f2ldlrex | 0.8 |
| c1hdl + f2ldl | 0.8 |
| c1hdl + c1ldlrex | 0.799 |
| cd14ldlhdl + f2rextex | 0.799 |
| cd14ldlhdl + ccldltex | 0.797 |
| cd14ldlhdl + cd14rextex | 0.797 |
| c1hdltex + cchdlrex | 0.794 |
| cdl4tex + cd14ldlhdl | 0.793 |
| cd14ldlhdl + cchdltex | 0.793 |
| cd14ldlhdl + g1tex | 0.792 |
| cd14hdlrex + c1tex | 0.791 |
| c1hdltex + f2ldl | 0.79 |
| cd14ldlhdl + cchdlrex | 0.789 |
| c1ldlhdl + c1hdltex | 0.787 |
| cd14hdltex + ccldlrex | 0.784 |
| cd14ldlhdl + ccrextex | 0.784 |
| cdl4hdlrex + c1ldlhdl | 0.783 |
| cd14ldlhdl + ccldlrex | 0.783 |
| cd14ldlhdl + g1hdl | 0.781 |
| cd14ldlhdl + ccldlhdl | 0.78 |
| cdl4hdlrex + f2ldlrex | 0.78 |
| cdl4hdl + cchdlrex | 0.779 |
| cd14ldlhdl + cd14hdlrex | 0.779 |
| cd14ldlhdl + cctex | 0.779 |
| cdl4hdltex + cd14rextex | 0.779 |
| cllex + c1ldlhdl | 0.778 |
| cd14ldlhdl + cd14ldlrex | 0.777 |
| cd14ldlhdl + c1ldlrex | 0.776 |
| cd14ldlhdl + c1ldltex | 0.776 |
| cd14ldlhdl + f2ldltex | 0.776 |
| cd14hdl + cd14rex | 0.774 |
| cd14hdltex + ccrex | 0.774 |
| cd14hdltex + f2hdltex | 0.774 |
| cd14ldl + cdl4hdl | 0.773 |
| cd14hdl + cd14ldlhdl | 0.773 |
| cd14hdl + c1tex | 0.772 |
| cd14rex + cdl4hdlrex | 0.772 |
| cd14ldlrex + cdl4hdlrex | 0.772 |
| cd14ldl + cd14ldlhdl | 0.772 |
| cd14hdlrex + g1rex | 0.771 |
| cd14hdltex + g1hdlrex | 0.771 |
| cd14ldlhdl + g1ldl | 0.771 |
| f2ldlrex + cchdlrex | 0.771 |
| cd14hdl + cd14hdlrex | 0.77 |
| c1hdltex + f2ldlrex | 0.77 |
| cd14ldlhdl + g1hdlrex | 0.769 |
| cd14ldlhdl + ccldl | 0.769 |
| cd14ldlhdl + f2rex | 0.769 |
| c1ldlhdl + g1tex | 0.768 |
| cd14ldlhdl + c1ldl | 0.768 |
| cd14ldlhdl + g1rex | 0.768 |
| cd14ldlhdl + f2hdl | 0.768 |
| c1ldlhdl + cchdlrex | 0.767 |
| cd14ldlhdl + g1ldlhdl | 0.766 |
| c1ldlhdl + f2ldlrex | 0.763 |
| cdl4rex + cd14ldlhdl | 0.762 |
| cd14ldlhdl + f2hdlrex | 0.762 |
| cd14ldlhdl + cchdl | 0.761 |
| cd14ldlhdl + g1ldlrex | 0.761 |
| cd14ldlhdl + ccrex | 0.76 |
| cd14hdltex + g1ldlrex | 0.76 |
| cd14hdl + cctex | 0.759 |
| cd14ldlhdl + c1rex | 0.758 |
| cd14ldlhdl + f2tex | 0.757 |
| cd14ldlhdl + f2hdltex | 0.757 |
| cd14hdlrex + cctex | 0.756 |
| cd14hdlrex + g1hdl | 0.752 |
| cd14hdltex + g1ldl | 0.752 |
| cd14tex + c1ldlhdl | 0.751 |
| cd14hdlrex + g1ldlrex | 0.751 |
| cd14hdlrex + f2hdlrex | 0.749 |
| cd14hdl + c1rextex | 0.748 |
| cd14hdlrex + g1rextex | 0.748 |
| cd14hdlrex + g1ldl | 0.747 |
| cd14hdlrex + g1tex | 0.747 |
| cd14hdlrex + c1rextex | 0.746 |
| cd14tex + cd14hdlrex | 0.746 |
| cd14hdlrex + cchdl | 0.746 |
| c1tex + g1hdl | 0.746 |
| cd14hdltex + c1rex | 0.744 |
| cd14hdltex + ccldl | 0.744 |
| cd14hdlrex + ccldlhdl | 0.744 |
| cd14hdlrex + f2hdl | 0.744 |
| cd14hdl + cdl4hdltex | 0.743 |
| cd14hdlrex + ccrex | 0.743 |
| cd14hdlrex + g1hdlrex | 0.743 |
| cd14ldlhdl + f2ldlrex | 0.743 |
| cd14ldl + c1ldlhdl | 0.742 |
| cd14hdl + f2rextex | 0.741 |
| c1ldlhdl + c1ldltex | 0.741 |
| cd14hdl + g1ldltex | 0.741 |
| cd14hdl + cchdltex | 0.741 |
| cd14hdlrex + f2rextex | 0.741 |
| cd14hdlrex + cd14hdltex | 0.741 |
| cd14hdlrex + ccrextex | 0.741 |
| c1tex + g1rex | 0.741 |
| cd14hdl + g1tex | 0.74 |
| cd14hdlrex + f2rex | 0.74 |
| cd14hdlrex + g1ldltex | 0.74 |
| cd14hdlrex + f2tex | 0.74 |
| cd14hdlrex + ccldltex | 0.74 |
| cd14ldl + cd14hdlrex | 0.739 |
| cd14hdlrex + c1rex | 0.739 |
| cd14hdlrex + cd14rextex | 0.739 |
| cd14hdl + g1hdltex | 0.738 |
| cd14hdl + g1ldlhdl | 0.738 |
| c1tex + c1ldltex | 0.738 |
| cd14hdl + g1rextex | 0.737 |
| cd14hdlrex + c1ldlrex | 0.737 |
| cd14hdlrex + ccldlrex | 0.737 |
| cd14ldltex + cd14hdlrex | 0.737 |
| cd14hdlrex + g1hdltex | 0.737 |
| c1ldlhdl + f21dl | 0.737 |
| cdl4hdltex + c1ldlhdl | 0.736 |
| f2hdl + f2tex | 0.736 |
| cd14hdlrex + g1ldlhdl | 0.736 |
| cd14hdlrex + cchdltex | 0.736 |
| c1ldlhdl + c1rextex | 0.734 |
| c1ldlhdl + cchdltex | 0.734 |
| cd14hdl + cd14rextex | 0.734 |
| cd14hdlrex + ccldl | 0.733 |
| c1tex + cchdlrex | 0.733 |
| cd14hdl + ccldl | 0.732 |
| cd14rex + c1ldlhdl | 0.732 |
| c1ldlhdl + cctex | 0.732 |
| cd14hdl + cd14ldltex | 0.732 |
| cd14hdl + ccldlrex | 0.732 |
| c1ldlhdl + f2hdlrex | 0.732 |
| cd14hdltex + 12rex | 0.732 |
| cd14hdl + g1hdl | 0.731 |
| c1ldlhdl + f2tex | 0.73 |
| c1rextex + g1rextex | 0.73 |
| c1rextex + ccrextex | 0.73 |
| cd14hdlrex + cchdlrex | 0.73 |
| cd14hdl + f2ldlrex | 0.73 |
| cd14ldlhdl + f2ldl | 0.73 |
| cd14hdl + ccldltex | 0.729 |
| cd14hdl + c1ldlhdl | 0.729 |
| cd14hdl + ccrex | 0.729 |
| cd14hdlrex + c1ldltex | 0.729 |
| cd14hdl + g1hdlrex | 0.729 |
| ccldltex + cchdltex | 0.729 |
| c1ldlhdl + ccrex | 0.728 |
| c1rex + c1ldlhdl | 0.728 |
| cdl4hdl + ccldlhdl | 0.728 |
| drextex + ccldltex | 0.728 |
| c1ldlhdl + g1hdl | 0.727 |
| c1tex + g1ldl | 0.727 |
| c1ldlhdl + g1ldlhdl | 0.726 |
| cdl4hdlrex + c1ldl | 0.726 |
| cdl4hdlrex + f2hdltex | 0.725 |
| cd14ldltex + c1ldlhdl | 0.724 |
| cdl4hdl + ccrextex | 0.724 |
| c1ldlhdl + f2rextex | 0.723 |
| c1tex + f2hdl | 0.723 |
| c1ldlhdl + g1hdlrex | 0.722 |
| c1ldlhdl + g1rextex | 0.722 |
| cd14hdl + c1ldl | 0.722 |
| cd14rextex + c1ldlhdl | 0.721 |
| cd14hdl + 12tex | 0.72 |
| cd14hdl + c1ldltex | 0.72 |
| c1ldlhdl + g1hdltex | 0.72 |
| c1ldlhdl + ccldltex | 0.72 |
| c1ldlhdl + ccrextex | 0.72 |
| c1ldlhdl + g1rex | 0.72 |
| c1ldlhdl + ccldlhdl | 0.72 |
| cd14hdl + f2hdlrex | 0.72 |
| c1tex + g1hdlrex | 0.72 |
| c1ldlhdl + g1ldltex | 0.719 |
| c1rextex + g1ldlhdl | 0.719 |
| cd14ldl + c1rextex | 0.719 |
| cd14hdl + f2hdltex | 0.718 |
| cd14hdl + cchdl | 0.718 |
| cd14hdltex + f2hdlrex | 0.718 |
| cd14hdl + f2ldltex | 0.718 |
| cd14hdlrex + f2ldl | 0.718 |
| c1ldlhdl + f2hdltex | 0.717 |
| c1rextex + g1hdltex | 0.717 |
| cchdl + cctex | 0.717 |
| cdl4hdl + g1ldlrex | 0.717 |
| c1tex + g1rextex | 0.717 |
| f2ldlrex + f2hdltex | 0.717 |
| c1ldlhdl + f2rex | 0.716 |
| c1ldlhdl + cchdl | 0.716 |
| c1rextex + g1ldltex | 0.716 |
| c1lex + c1ldlrex | 0.716 |
| f21dlrex + ccldlrex | 0.716 |
| cdl4hdlrex + f2ldltex | 0.716 |
| cdl4hdltex + g1rex | 0.715 |
| cdl4hdl + cd14ldlrex | 0.714 |
| cdl4hdl + c1ldlrex | 0.714 |
| c1rextex + ccrex | 0.714 |
| cdl4hdl + f2hdl | 0.714 |
| c1ldlhdl + f2ldltex | 0.714 |
| c1tex + g1tex | 0.713 |
| c1ldlhdl + g1ldl | 0.713 |
| cdl4hdl + f2ldl | 0.713 |
| c1ldlhdl + ccldl | 0.712 |
| cd14ldl + c1tex | 0.712 |
| cd14ldlrex + c1ldlhdl | 0.712 |
| drextex + ccldlrex | 0.712 |
| cdl4hdl + c1rex | 0.71l |
| cdl4hdl + g1ldl | 0.71l |
| c1rextex + f2hdltex | 0.71l |
| cdl4rextex + c1tex | 0.71l |
| cdl4tex + c1tex | 0.71l |
| cdl4hdl + g1rex | 0.71 |
| cd14ldltex + c1tex | 0.71 |
| c1rex + c1tex | 0.71 |
| c1tex + ccldlhdl | 0.71 |
| c1ldlhdl + ccldlrex | 0.709 |
| cdl4hdl + f2rex | 0.709 |
| c1tex + f2rex | 0.709 |
| c1tex + g1ldlhdl | 0.709 |
| cd14ldlrex + c1tex | 0.709 |
| c1lex + g1ldlrex | 0.709 |
| cd14ldltex + c1rextex | 0.709 |
| c1rextex + g1hdlrex | 0.709 |
| f2ldl + f2rex | 0.709 |
| ccrex + cchdlrex | 0.709 |
| g1hdl + cchdlrex | 0.708 |
| cdl4hdltex + c1tex | 0.707 |
| c1tex + ccrex | 0.707 |
| cdl4hdltex + cctex | 0.707 |
| c1tex + ccldltex | 0.706 |
| c1ldlhdl + f2hdl | 0.706 |
| cdl4rex + c1tex | 0.706 |
| c1ldl + c1tex | 0.706 |
| c1lex + f2ldlrex | 0.706 |
| c1ldlrex + f2ldlrex | 0.705 |
| cd14rex + cd14hdltex | 0.704 |
| c1tex + c1rextex | 0.704 |
| c1tex + cctex | 0.704 |
| c1rex + c1rextex | 0.703 |
| c1tex + f2rextex | 0.703 |
| c1tex + ccrextex | 0.703 |
| c1ldltex + f2hdltex | 0.703 |
| cdl4hdltex + c1ldl | 0.703 |
| f2rextex + cchdlrex | 0.703 |
| f21dlrex + g1hdlrex | 0.703 |
| f21dlrex + g1hdl | 0.702 |
| cd14hdltex + ccldlhdl | 0.701 |
| c1tex + g1ldltex | 0.701 |
| c1tex + g1hdltex | 0.701 |
| c1tex + cchdl | 0.701 |
| cllex + ccldlrex | 0.701 |
| c1tex + cchdltex | 0.701 |
| c1ldlhdl + g1ldlrex | 0.701 |
| c1rextex + ccldl | 0.701 |
| g1rex + cchdlrex | 0.7 |
| cd14hdltex + f2hdl | 0.699 |
| c1rextex + g1ldl | 0.699 |
| c1tex + f2hdlrex | 0.699 |
| cdl4tex + cd14hdltex | 0.699 |
| cdl4hdltex + c1ldltex | 0.699 |
| c1tex + f2hdltex | 0.699 |
| c1tex + f2tex | 0.698 |
| c1lex + ccldl | 0.698 |
| cd14hdltex + cchdlrex | 0.698 |
| f2ldlrex + cchdl | 0.698 |
| f2ldlrex + g1tex | 0.698 |
| cdl4tex + cchdl | 0.697 |
| g1rextex + cchdltex | 0.697 |
| cd14ldlrex + f2ldlrex | 0.697 |
| f2hdl + f21dlrex | 0.697 |
| cd14hdltex + c1rextex | 0.696 |
| ccldlrex + cchdlrex | 0.696 |
| c1ldltex + f2ldlrex | 0.696 |
| f2ldl + f2hdltex | 0.696 |
| cchdl + cchdlrex | 0.694 |
| c1rextex + cchdlrex | 0.694 |
| cd14hdltex + f2ldlrex | 0.694 |
| cd14hdltex + f2ldltex | 0.694 |
| c1ldl + f21dlrex | 0.693 |
| c1rextex + f2ldlrex | 0.693 |
| f2ldlrex + g1hdltex | 0.693 |
| cdl4hdltex + cchdl | 0.692 |
| c1rextex + f2hdl | 0.692 |
| c1rextex + ccldlhdl | 0.692 |
| cchdltex + ccrextex | 0.692 |
| f2rex + f2ldlrex | 0.692 |
| f21dlrex + g1ldltex | 0.692 |
| f2rextex + cchdl | 0.691 |
| c1rextex + cctex | 0.691 |
| cdl4tex + c1rextex | 0.69 |
| g1rextex + ccldlhdl | 0.69 |
| f2ldl + f2ldlrex | 0.69 |
| g1rextex + cchdlrex | 0.69 |
| cd14hdltex + cchdltex | 0.69 |
| cd14rex + cchdlrex | 0.689 |
| f2ldlrex + ccldlhdl | 0.689 |
| ccldlhdl + ccldlrex | 0.689 |
| cdl4rex + f21dlrex | 0.689 |
| f2rextex + ccldlhdl | 0.688 |
| g1hdl + cchdl | 0.688 |
| cdl4rextex + c1rextex | 0.688 |
| f2ldlrex + f2ldltex | 0.688 |
| f2ldlrex + ccrextex | 0.688 |
| f2ldlrex + g1ldl | 0.688 |
| f2ldlrex + g1rex | 0.688 |
| f2ldlrex + g1ldlrex | 0.688 |
| cdl4rextex + f2ldlrex | 0.687 |
| f2ldlrex + ccldl | 0.687 |
| g1ldlhdl + cchdltex | 0.686 |
| ccldlhdl + cchdltex | 0.686 |
| f21dlrex + cchdltex | 0.685 |
| cd14ldl + f2ldlrex | 0.685 |
| c1rex + f2ldlrex | 0.685 |
| f2ldlrex + g1ldlhdl | 0.685 |
| f2ldlrex + ccrex | 0.685 |
| f2ldlrex + g1rextex | 0.685 |
| g1hdltex + cchdltex | 0.684 |
| g1tex + cchdlrex | 0.684 |
| f2ldlrex + f2rextex | 0.684 |
| cd14ldltex + f2ldlrex | 0.684 |
| f2ldlrex + ccldltex | 0.684 |
| g1ldltex + cchdltex | 0.683 |
| cd14ldltex + cchdltex | 0.683 |
| cdl4tex + f2ldlrex | 0.683 |
| f2tex + f2ldlrex | 0.683 |
| ccldlrex + cchdltex | 0.681 |
| c1ldlrex + cchdlrex | 0.681 |
| c1rextex + f2rex | 0.681 |
| c1rextex + g1tex | 0.681 |
| f2hdltex + g1rextex | 0.681 |
| cd14ldl + cchdltex | 0.68 |
| c1rextex + g1ldlrex | 0.68 |
| g1ldltex + ccldlhdl | 0.679 |
| cd14ldlrex + cdl4hdltex | 0.679 |
| g1tex + ccldlhdl | 0.678 |
| c1rextex + g1rex | 0.678 |
| g1ldlrex + cchdlrex | 0.678 |
| c1rextex + cchdl | 0.677 |
| cd14rex + c1rextex | 0.677 |
| f2tex + cchdlrex | 0.677 |
| cchdlrex + ccrextex | 0.677 |
| f2ldlrex + cctex | 0.677 |
| cchdlrex + cchdltex | 0.677 |
| f2ldltex + f2rextex | 0.677 |
| g1hdltex + ccldlhdl | 0.676 |
| c1rextex + f2hdlrex | 0.675 |
| cd14hdltex + f2rextex | 0.674 |
| cd14tex + ccldlhdl | 0.674 |
| ccldlhdl + ccldltex | 0.674 |
| cchdl + ccrex | 0.673 |
| c1ldl + cchdlrex | 0.673 |
| ccldlhdl + cchdlrex | 0.673 |
| c1rextex + 12rextex | 0.672 |
| cd14rextex + ccldlhdl | 0.672 |
| cd14ldltex + ccldlhdl | 0.672 |
| cd14ldlrex + cchdlrex | 0.672 |
| cd14hdltex + 121dl | 0.672 |
| f2ldl + cchdlrex | 0.672 |
| f2hdltex + g1hdltex | 0.671 |
| ccldl + cchdl | 0.671 |
| cctex + ccldlhdl | 0.671 |
| cdl4hdltex + f2tex | 0.671 |
| cdl4tex + cchdlrex | 0.671 |
| cdl4hdltex + c1ldlrex | 0.67 |
| cdl4hdltex + g1tex | 0.67 |
| cd14ldlrex + c1rextex | 0.67 |
| cdl4rex + ccldlhdl | 0.669 |
| ccldlhdl + ccrextex | 0.669 |
| g1ldl + cchdltex | 0.668 |
| g1ldlrex + cchdltex | 0.668 |
| c1lex + f21dl | 0.668 |
| f2ldltex + cchdlrex | 0.668 |
| cdl4rextex + cchdlrex | 0.667 |
| f2ldltex + cchdl | 0.667 |
| f2tex + g1hdl | 0.666 |
| f2hdl + f2rextex | 0.666 |
| g1ldl + cchdlrex | 0.666 |
| c1lex + f2ldltex | 0.665 |
| ccldl + ccldlhdl | 0.664 |
| f2hdl + ccldlhdl | 0.664 |
| cd14ldltex + f2rextex | 0.663 |
| cdl4rextex + f2rextex | 0.663 |
| f2rextex + ccrextex | 0.663 |
| c1ldl + c1rextex | 0.663 |
| g1ldlrex + ccldlhdl | 0.663 |
| g1hdlrex + cchdlrex | 0.663 |
| cd14rextex + ccrex | 0.662 |
| c1rex + cchdltex | 0.662 |
| c1rex + cchdlrex | 0.662 |
| g1ldltex + cchdl | 0.661 |
| g1rextex + cchdl | 0.661 |
| g1ldlhdl + cchdlrex | 0.661 |
| f2rextex + ccldltex | 0.66 |
| cchdl + cchdltex | 0.66 |
| f2rex + ccldlhdl | 0.66 |
| cd14ldl + ccldlhdl | 0.66 |
| ccrex + cchdltex | 0.66 |
| c1ldl + ccldlhdl | 0.659 |
| g1ldl + ccldlhdl | 0.659 |
| g1rex + g1tex | 0.659 |
| g1hdltex + cchdl | 0.657 |
| g1tex + cchdl | 0.657 |
| c1rextex + 12tex | 0.657 |
| g1hdlrex + cchdl | 0.657 |
| f2hdltex + g1ldltex | 0.656 |
| f2hdltex + cchdl | 0.656 |
| f2ldltex + ccldlhdl | 0.656 |
| f2ldltex + f2hdlrex | 0.655 |
| c1rextex + 121dl | 0.655 |
| g1rex + ccldlhdl | 0.654 |
| g1rex + cchdltex | 0.654 |
| cd14ldl + cchdlrex | 0.654 |
| f2hdl + cchdlrex | 0.654 |
| f2hdlrex + cchdlrex | 0.654 |
| cd14ldltex + cchdl | 0.653 |
| cdl4hdltex + g1hdl | 0.653 |
| c1ldltex + ccldlhdl | 0.653 |
| f2tex + cchdl | 0.653 |
| cdl4rex + cchdl | 0.653 |
| cchdl + ccldlrex | 0.653 |
| f2hdltex + ccldlhdl | 0.653 |
| g1ldltex + cchdlrex | 0.653 |
| g1hdlrex + cchdltex | 0.653 |
| cdl4rextex + cchdl | 0.652 |
| cdl4tex + cchdltex | 0.652 |
| c1ldltex + cctex | 0.652 |
| g1ldl + cchdl | 0.651 |
| ccrex + ccldlhdl | 0.651 |
| f2hdl + cchdltex | 0.651 |
| f2hdltex + cchdltex | 0.651 |
| g1ldl + g1rextex | 0.651 |
| g1hdlrex + ccldlhdl | 0.651 |
| c1ldltex + cchdlrex | 0.651 |
| cctex + cchdltex | 0.65 |
| f2tex + ccldlhdl | 0.65 |
| cctex + cchdlrex | 0.65 |
| ccldl + cchdltex | 0.65 |
| cdl4rextex + g1hdltex | 0.649 |
| g1hdl + ccldlhdl | 0.649 |
| c1rex + ccldlhdl | 0.649 |
| cd14ldlrex + ccldlhdl | 0.649 |
| f2rex + cchdltex | 0.649 |
| cchdl + ccldltex | 0.648 |
| g1ldl + g1tex | 0.648 |
| ccldltex + cchdlrex | 0.648 |
| g1hdlrex + cctex | 0.648 |
| cd14ldltex + cchdlrex | 0.648 |
| f2rextex + g1ldl | 0.647 |
| f2tex + g1ldl | 0.647 |
| g1hdl + cctex | 0.647 |
| f2tex + 12hdlrex | 0.646 |
| g1ldlhdl + ccldlhdl | 0.646 |
| f2hdltex + ccldlrex | 0.646 |
| f2tex + g1tex | 0.644 |
| g1hdl + g1tex | 0.644 |
| cchdl + ccldlhdl | 0.644 |
| c1ldlrex + g1tex | 0.642 |
| g1ldlhdl + cchdl | 0.642 |

The three best individual markers appeared to be SerpinC1 with the ratio HDL/TEX and an AUC of 0.842 (95% Cl 0.741-0.944); SerpinC1 with the ratio HDL/REX and an AUC of 0.828 (95% Cl 0.718-0.938)) and SerpinC1 in the HDL fraction with an AUC of 0.812 (95% Cl 0.700-0.924) to discriminate between stable coronary artery disease and matched controls. Selection of the best combination of markers and sub-fractions (without the ratios of markers between the sub-fractions) using Logistics regression analysis with forward selection based upon Akaike Information Criterion (AIC) showed an AUC of 0.861 (95% Cl 0.86-1). The combination of markers consisted of SerpinC1-HDL+SerpinG1-TEX+SerpinC1-REX (see FIG. 4) without using the ratios between the sub-fractions, because with 30 vs 30 patients one is underpowered to use all biomarkers in all combinations. This is a conservative choice that prevents overfitting of the regression model and for this gives a more realistic calculation. These results show that using the same set of extracellular protein levels in four fractions can be used in a different cohort of a different cardiovascular syndrome: stable angina, which has the same underlying disease process: atherosclerosis. Using the selected combination with an optimal cut-off value based on the AUC, it was shown that the sensitivity of these three markers was 93.3% and specificity of 76.7%. The NPV is 92 and the Positive Predictive Value is 80%. FIG. 5 shows the highest scoring combinations of marker/sub-fraction for the Myomarker cohort but now (as for unstable angina) including the ratios of the biomarker between the plasma sub-fractions. This combination/ratios between fractions is used in the calculation of the best combination of extracellular vesicle markers after validation of the extracellular vesicle markers in the large cohorts (Example 5) that will give enough power as patient numbers are much higher.

### Example 5. Validation of Extracellular Vesicle Proteins for Diagnosis of Stable Angina and Unstable Angina in Large Cohorts

### Diagnosis of Unstable Angina

A follow up study is executed for the validation of the extracellular vesicle proteins for the diagnosis of unstable angina. In the MINERVA cohorts, SerpinF2, SerpinG1, SerpinC1, CD14 and Cystatin C are measured in all plasma fractions of LDL, HDL, REX, TEX using sequential precipitation and an immuno-bead assay as described above. In the MINERVA cohort, also described above, more than 2000 patients with suspected acute coronary syndrome were included on the emergency department, including 100 patients with unstable angina. Using back and forward selection, the AUC, NPV, PPV, sensitivity and specificity are determined in order to identify the markers and the combination of markers (including the ratios of the biomarker between the plasma sub-fractions) with the best diagnostic performance.

### Diagnosis of Stable Angina

In addition, a follow up study is executed for the validation of the extracellular vesicle proteins for the diagnosis of stable angina. For this, the MYOMARKER cohort, described above, is used. This cohort contains around 1250 patients with suspected myocardial ischemia causing stable angina. It is anticipated that around 250 patients have significant myocardial ischemia on radionuclide myocardial perfusion imaging. In all patients, the 5 extracellular vesicle proteins SerpinF2, SerpinG1, SerpinC1, CD14 and Cystatin C are measured in the plasma fractions LDL, HDL, REX and TEX using sequential precipitation and an immuno-bead assay. Using back and forward selection, the AUC, NPV, PPV, sensitivity and specificity are determined in order to identify the markers (including the ratios of the biomarker between the plasma sub-fractions) and the combination of markers with the best diagnostic performance.

1. A method for identifying a subject suffering from, or being at risk of suffering from, an ischemic heart disease, said method comprising the steps of:
a) performing sequential precipitation with polyanions to obtain one or more of the plasma fractions selected from the group consisting of the Low-Density Lipoprotein (LDL) fraction, the High-Density Lipoprotein (HDL) fraction, the remaining (REX) fraction and the total (TEX) fraction from a plasma sample of said subject;
b) performing an immune-bead assay to measure the concentration of one or more protein marker in one or more of said plasma fractions, wherein the at least one or more protein marker is selected from SerpinC1, Cystatin C, CD14, SerpinF2 and SerpinG1;
c) computing one or more values, wherein each of the one or more values:
is derived from a concentration of a protein marker in one of said plasma fractions, and/or
is a ratio of the concentrations of a single protein marker in two different ones of said plasma fractions,
d) performing a comparison of the one or more values as determined in step c) with one or more corresponding reference values, which has been derived in the same way from the concentration of the same one or more protein marker in corresponding plasma fractions as determined in group of reference subjects not suffering from ischemic heart disease, wherein a statistically significant difference between the one or more values determined in step c) and the one or more corresponding reference values is indicative of the subject suffering, or being at risk of suffering, from an ischemic heart disease.

2. The method according to claim 1, wherein the one or more values in step c) are selected such that the area under the curve (AUC) differs from the diagonal reference line of 0.5 with a p-value of 0.05 or less, as determined by Receiver Operating Characteristic (ROC) plot analysis of the combination of said one or more values based on a suitable group of definitive subjects and group of reference subjects.

3. The method according to claim 1, wherein the one or more values in step c) are selected such that the area under the curve (AUC) is 0.8 or more and the p-value is 0.05 or less, as determined by Receiver Operating Characteristic (ROC) plot analysis of the combination of said one or more values based on a suitable group of definitive subjects and group of reference subjects.

4. The method according to claim 1, wherein the negative predictive value and/or the positive predictive value is 0.8 or more.

5. The method according to claim 1, wherein said ischemic heart disease is unstable angina.

6. The method according to claim 5, wherein at least one of the one or more values is selected from the group consisting of values derived from the concentration of:
SerpinC1 in the HDL plasma fraction,
CD14 in the HDL plasma fraction,
CD14 in the REX plasma fraction,
CD14 in the TEX plasma fraction, and
SerpinF2 in the TEX plasma fraction,
and/or wherein at least one of the one or more values is a ratio of two values derived from concentrations of a single protein marker in two different ones of said plasma fractions, selected from the group consisting of:
the concentration of SerpinC1 in the LDL plasma fraction over the concentration in the HDL plasma fraction,
the concentration of SerpinC1 in the HDL plasma fraction over the concentration in the REX plasma fraction,
the concentration of SerpinC1 in the HDL plasma fraction over the concentration in the TEX plasma fraction,
the concentration of CD14 in the LDL plasma fraction over the concentration in the HDL plasma fraction,
the concentration of CD14 in the LDL plasma fraction over the concentration in the TEX plasma fraction,
the concentration of CD14 in the HDL plasma fraction over the concentration in the REX plasma fraction,
the concentration of CD14 in the HDL plasma fraction over the concentration in the TEX plasma fraction,
the concentration of Cystatin C in the LDL plasma fraction over the concentration in the HDL plasma fraction,
the concentration of Cystatin C in the HDL plasma fraction over the concentration in the REX plasma fraction,
the concentration of Cystatin C in the HDL plasma fraction over the concentration in the TEX plasma fraction, and
the concentration of SerpinF2 in the REX plasma fraction over the concentration in the TEX plasma fraction.

7. The method according to claim 6, wherein at least one of the one or more values is selected from the group consisting of values derived from the concentration of:
CD14 in the REX plasma fraction,
CD14 in the TEX plasma fraction, and
SerpinF2 in the TEX plasma fraction,
and/or wherein at least one of the one or more values is a ratio of two values derived from concentrations of a single protein marker in two different ones of said plasma fractions, selected from the group consisting of:
the concentration of SerpinC1 in the LDL plasma fraction over the concentration in the HDL plasma fraction,
the concentration of CD14 in the LDL plasma fraction over the concentration in the HDL plasma fraction,
the concentration of CD14 in the LDL plasma fraction over the concentration in the TEX plasma fraction,
the concentration of CD14 in the HDL plasma fraction over the concentration in the REX plasma fraction, and
the concentration of SerpinF2 in the REX plasma fraction over the concentration in the TEX plasma fraction.

8. The method according to claim 1, wherein said ischemic heart disease is stable angina.

9. The method according to claim 8, wherein at least one of the one or more values is selected from the group consisting of values derived from the concentration of:
SerpinC1 in the HDL plasma fraction,
SerpinC1 in the TEX plasma fraction, and
CD14 in the HDL plasma fraction,
and/or wherein at least one of the one or more values is a ratio of two values derived from concentrations of a single protein marker in two different ones of said plasma fractions, selected from the group consisting of:
the concentration of SerpinC1 in the LDL plasma fraction over the concentration in the HDL plasma fraction,
the concentration of SerpinC1 in the HDL plasma fraction over the concentration in the REX plasma fraction,
the concentration of SerpinC1 in the HDL plasma fraction over the concentration in the TEX plasma fraction,
the concentration of SerpinC1 in the REX plasma fraction over the concentration in the TEX plasma fraction,
the concentration of CD14 in the LDL plasma fraction over the concentration in the HDL plasma fraction,
the concentration of CD14 in the HDL plasma fraction over the concentration in the REX plasma fraction,
the concentration of CD14 in the HDL plasma fraction over the concentration in the TEX plasma fraction,
the concentration of Cystatin C in the LDL plasma fraction over the concentration in the HDL plasma fraction,
the concentration of Cystatin C in the HDL plasma fraction over the concentration in the REX plasma fraction,
the concentration of Cystatin C in the HDL plasma fraction over the concentration in the TEX plasma fraction,
the concentration of SerpinF2 in the LDL plasma fraction over the concentration in the HDL plasma fraction,
the concentration of SerpinF2 in the LDL plasma fraction over the concentration in the REX plasma fraction,
the concentration of SerpinF2 in the HDL plasma fraction over the concentration in the TEX plasma fraction, and
the concentration of SerpinG1 in the REX plasma fraction over the concentration in the TEX plasma fraction.

10. The method according to claim 9, wherein at least one of the one or more values is selected from the group consisting of values derived from the concentration of:
SerpinC1 in the TEX plasma fraction,
and/or wherein at least one of the one or more values is a ratio of two values derived from concentrations of a single protein marker in two different ones of said plasma fractions, selected from the group consisting of:
the concentration of SerpinC1 in the REX plasma fraction over the concentration in the TEX plasma fraction,
the concentration of SerpinF2 in the LDL plasma fraction over the concentration in the HDL plasma fraction,
the concentration of SerpinF2 in the LDL plasma fraction over the concentration in the REX plasma fraction,
the concentration of SerpinF2 in the HDL plasma fraction over the concentration in the TEX plasma fraction, and
the concentration of SerpinG1 in the REX plasma fraction over the concentration in the TEX plasma fraction.

11. The method according to claim 1, wherein at least one of the one or more values is a ratio of two values derived from concentrations of a single protein marker in two different ones of said plasma fractions, wherein the protein marker is selected from the group consisting of SerpinC1, Cystatin C, CD14, SerpinF2 and SerpinG1.

12. The method according to claim 1, wherein step comprises determining at least two values involving at least two different protein markers.

13. The method according to claim 1, wherein at least one protein marker is SerpinC1.

14. The method according to claim 13, wherein at least one further protein marker is selected from the group consisting of Cystatin C, CD14, SerpinF2 and SerpinG1.

15. A kit comprising means for performing the method of claim 1.
